(19) United States
(12) Patent Application Publication
(10) Pub. No.: US 2021/0322348 A1
(43) Pub. Date: Oct. 21, 2021

(54) METHODS OF ADMINISTERING GAMMA-HYDROXYBUTYRATE COMPOSITIONS WITH DIVALPROEX SODIUM

(71) Applicant: Flamel Ireland Limited, Dublin (IE)

(72) Inventor: Julien Grassot, Lyon (FR)

(21) Appl. No.: 17/231,455

(22) Filed: Apr. 15, 2021

Related U.S. Application Data
(60) Provisional application No. 63/010,974, filed on Apr. 16, 2020.

(51) Int. Cl. A61K 31/19; A61K 9/00

(52) U.S. Cl. CPC A61K 31/19; A61K 9/0053

(57) ABSTRACT

Oral pharmaceutical compositions of gamma-hydroxybutyrate (GHB) suitable for concomitant administration with a dose of divalproex sodium (DVP) without materially altering the dosage amount of either drug are provided. Also provided are therapeutic uses of the compositions for the treatment of one or more symptoms of narcolepsy.

### CROSS-REFERENCE TO RELATED APPLICATIONS

This application claims priority to U.S. Provisional Application No. 63/010,974, filed Apr. 16, 2020.

### FIELD

The present invention relates to compositions for the treatment of narcolepsy, such as any of the symptoms of narcolepsy (e.g., cataplexy, excessive daytime sleepiness, disrupted nighttime sleep, hypnagogic hallucinations, or sleep paralysis) comprising gamma-hydroxybutyrate in a unit dose suitable for administration with divalproex sodium. The present invention also relates to modified release formulations of gamma-hydroxybutyrate having improved pharmacokinetic (PK) properties with concomitant administration of divalproex sodium.

### BACKGROUND

Narcolepsy is a devastating disabling condition. The cardinal symptoms are excessive daytime sleepiness (EDS), cataplexy (a sudden loss of muscle tone triggered by strong emotions, seen in approximately 60% of patients), hypnogogic hallucination (HH), sleep paralysis (SP), and disturbed nighttime/nocturnal sleep (DNS). Other than EDS, DNS is the most common symptom seen among narcolepsy patients.

One of the major treatments for narcolepsy is sodium oxybate, a neuroactive agent with a variety of Central Nervous System (CNS) pharmacological properties. The species is present endogenously in many tissues, where it acts as a neurotransmitter on a gamma-hydroxybutyrate (GHB) receptor (GHBR), and possesses neuromodulatory properties with significant effects on dopamine and gamma-Aminobutyric Acid (GABA). Studies have suggested that sodium oxybate improves Rapid Eye Movement Sleep (REM sleep, REMS) of narcoleptics in contrast to antidepressant drugs.

Sodium oxybate is also known as sodium 4-hydroxybutanoate, or gamma-hydroxybutyric acid sodium salt, and has the following chemical structure:

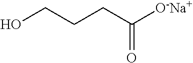

Sodium oxybate is marketed commercially in the United States as Xyrem®. The product is formulated as an immediate release liquid solution that is taken once immediately before bed, and a second time approximately 2.5 to 4 hours later, in equal doses. Sleep-onset may be dramatic and fast, and patients are advised to be sitting in bed when consuming the dose. The most commonly reported side effects are confusion, depressive syndrome, incontinence and sleepwalking.

One critical drawback of Xyrem® is the requirement to reduce the initial dosage of Xyrem if there is concomitant use with divalproex sodium (DVP). Specifically, Xyrem®'s label expressly advises “Concomitant use with Divalproex Sodium: an initial reduction in Xyrem® dose of at least 20% is recommended.” After a clinical trial for co-administration of Xyrem and divalproex sodium, the following language was added to the Xyrem label at section 2.4: “Pharmacokinetic and pharmacodynamic interactions have been observed when Xyrem is co administered with divalproex sodium. For patients already stabilized on Xyrem, it is recommended that addition of divalproex sodium should be accompanied by an initial reduction in the nightly dose of Xyrem by at least 20%. For patients already taking divalproex sodium, it is recommended that prescribers use a lower starting Xyrem dose when introducing Xyrem.” The medical problem cautioned against by the Xyrem® label and unaddressed by the prior art is pharmacokinetic and pharmacodynamic interactions when Xyrem® is co-administered with divalproex sodium. As noted in the Xyrem®'s Drug Interactions section of the Prescribing Information, “Concomitant use of Xyrem with divalproex sodium resulted in a 25% mean increase in systemic exposure to Xyrem (AUC ratio range of 0.8 to 1.7) and in a greater impairment on some tests of attention and working memory.” As a practical matter, this requires prescribers to monitor patient response closely and adjust dose accordingly for concomitant use of Xyrem® and divalproex sodium. In addition, U.S. Pat. No. 8,772,306 to Jazz Pharmaceuticals teaches that the dosage amount of GHB must be decreased by at least 5% decrease when the patient is receiving a concomitant administration of valproate, an acid, salt, or mixture thereof (e.g. divalproex sodium).

Accordingly, there is a need for compositions of gamma-hydroxybutyrate that can be co-administered with divalproex sodium without having to reduce the dose of gamma-hydroxybutyrate and without compromising safety or efficacy.

### SUMMARY OF THE INVENTION

In an aspect, the present disclosure encompasses a method of treating narcolepsy (e.g., one or more symptoms of narcolepsy) by administering a GHB composition concomitantly with divalproex sodium (DVP) without reducing the dose of GHB. For example, a method for treating a patient suffering from excessive daytime sleepiness (EDS), disrupted nighttime sleep (DNS), cataplexy, hypnagogic hallucinations, or sleep paralysis may include orally administering to the patient a full dosage amount of a pharmaceutical composition comprising GHB and concomitantly administering a full dosage amount of a pharmaceutical composition comprising DVP. In some examples, the dosage of the GHB composition is not reduced in response to the concomitant administration of DVP and/or the dosage of the DVP is not reduced in response to the concomitant administration of GHB composition. In other examples, where the dosage of one or both GHB and DVP is reduced, such reduction is by less than 5% of the full dosage amount in response to the concomitant administration of DVP.

Further provided herein is an oral pharmaceutical composition of GHB for the treatment of narcolepsy (e.g., one or more symptoms of narcolepsy) that may be concomitantly administered with DVP. In some examples, the dosage of the GHB composition is not reduced in response to the concomitant administration of DVP, and the dosage of the DVP is not reduced in response to the concomitant administration of GHB composition. In other words, both the dosage amounts of the GHB composition and the DVP are not reduced at all when coadministered. In other examples, the dosage of one or both the GHB composition and the DVP is reduced by less than 5% of the full dosage amount when coadministered.

Other aspects and iterations of the invention are described more thoroughly below.

### DESCRIPTION OF THE FIGURES

The patent or application file contains at least one drawing executed in color. Copies of this patent or patent application with color drawing(s) will be provided by the Office by request and payment of the necessary fee.

The accompanying drawings, which are incorporated in and constitute a part of this specification, illustrate several embodiments of the invention and together with the description serve to explain the principles of the invention.

FIG. 1A is a mean concentration versus time curve for 6 g FT218 administered alone and with DVP in the evening.

FIG. 1B is a series of individual profiles in a mean concentration versus time curve for 6 g FT218 administered alone and with DVP in the evening.

FIG. 2A shows a comparison of mean Tₘₐₓ for 6 g FT218 administered alone and with DVP in the evening.

FIG. 2B shows a comparison of mean Cₘₐₓ for 6 g FT218 administered alone and with DVP in the evening.

FIG. 2C shows a comparison of mean AUC_{inf} for 6 g FT218 administered alone and with DVP in the evening.

FIG. 3A is a mean concentration versus time curve for DVP administered alone and with FT218 in the evening.

FIG. 3B is a series of individual profiles in a mean concentration versus time curve for DVP administered alone and with FT218 in the evening.

FIG. 4A is a mean concentration versus time curve for 6 g FT218 administered alone and with DVP in the morning.

FIG. 4B is a series of individual profiles in a mean concentration versus time curve for 6 g FT218 administered alone and with DVP in the morning.

FIG. 5A shows a comparison of mean Tₘₐₓ for 6 g FT218 administered alone and with DVP in the morning.

FIG. 5B shows a comparison of mean Cₘₐₓ for 6 g FT218 administered alone and with DVP in the morning.

FIG. 5C shows a comparison of mean AUC_{inf} for 6 g FT218 administered alone and with DVP in the morning.

FIG. 6A is a mean concentration versus time curve for DVP administered alone and with FT218 in the morning.

FIG. 6B is a series of individual profiles in a mean concentration versus time curve for DVP administered alone and with FT218 in the morning.

FIG. 7 is a mean concentration versus time curve for 6 g FT218 administered alone and with DVP either in the morning (DDI #1) or in the evening (DDI #2).

### DETAILED DESCRIPTION

The present invention may be understood more readily by reference to the following detailed description of embodiments of the formulation, methods of treatment using some embodiments of the formulation, and the Examples included therein.

### Definitions and Use of Terms

Wherever an analysis or test is required to understand a given property or characteristic recited herein, it will be understood that the analysis or test is performed in accordance with applicable guidances, draft guidances, regulations and monographs of the United States Food and Drug Administration (“FDA”) and United States Pharmacopoeia (“USP”) applicable to drug products in the United States in force as of Nov. 1, 2015 unless otherwise specified. Clinical endpoints may be judged with reference to standards adopted by the American Academy of Sleep Medicine, including standards published at C Iber, S Ancoli-Israel, A Chesson, S F Quan. The AASM Manual for the Scoring of Sleep and Associated Events. Westchester, Ill.: American Academy of Sleep Medicine; 2007.

When a pharmacokinetic comparison is made between a formulation described or claimed herein and a reference product, it will be understood that the comparison is performed in a suitable designed cross-over trial, although it will also be understood that a cross-over trial is not required unless specifically stated. It will also be understood that the comparison may be made either directly or indirectly. For example, even if a formulation has not been tested directly against a reference formulation, it can still satisfy a comparison to the reference formulation if it has been tested against a different formulation, and the comparison with the reference formulation may be deduced therefrom.

As used in this specification and in the claims which follow, the singular forms “a,” “an” and “the” include plural referents unless the context dictates otherwise. Thus, for example, reference to “an ingredient” includes mixtures of ingredients, reference to “an active pharmaceutical agent” includes more than one active pharmaceutical agent, and the like.

“Bioavailability” means the rate and extent to which the active ingredient or active moiety is absorbed from a drug product and becomes available at the site of action.

“Relative bioavailability” or “Rel BA” or “RBA” means the percentage of mean AUC_{inf} of the tested product relative to the mean AUC_{inf} of the reference product for an equal total dose. Unless otherwise specified, relative bioavailability refers to the percentage of the mean AUC_{inf} observed for a full dose of the test product co-administered with divalproex sodium relative to the mean AUC_{inf} observed for an equal total dose of the test product without administration of divalproex sodium.

“Bioequivalence” means the absence of a significant difference in the rate and extent to which the active ingredient or active moiety in pharmaceutical equivalents or pharmaceutical alternatives become available at the site of drug action when administered at the same molar dose under similar conditions in an appropriately designed study. In some examples, “bioequivalence range” means a test composition/condition has a PK value within 80%-125% of the PK value for a reference composition/condition.

When ranges are given by specifying the lower end of a range separately from the upper end of the range, it will be understood that the range may be defined by selectively combining any one of the lower end variables with any one of the upper end variables that is mathematically and physically possible. Thus, for example, if a formulation may contain from 1 to 10 weight parts of a particular ingredient, or 2 to 8 parts of a particular ingredient, it will be understood that the formulation may also contain from 2 to 10 parts of the ingredient. In like manner, if a formulation may contain greater than 1 or 2 weight parts of an ingredient and up to 10 or 9 weight parts of the ingredient, it will be understood that the formulation may contain 1-10 weight parts of the ingredient, 2-9 weight parts of the ingredient, etc. unless otherwise specified, the boundaries of the range (lower and upper ends of the range) are included in the claimed range.

When used herein the term “about” or “substantially” or “approximately” will compensate for variability allowed for in the pharmaceutical industry and inherent in pharmaceutical products, such as differences in product strength due to manufacturing variation and time-induced product degradation. The term allows for any variation which in the practice of pharmaceuticals would allow the product being evaluated to be considered bioequivalent to the recited strength, as described in FDA's March 2003 Guidance for Industry on BIOAVAILABILITY AND BIOEQUIVALENCE STUDIES FOR ORALLY ADMINISTERED DRUG PRODUCTS—GENERAL CONSIDERATIONS.

When used herein the term “gamma-hydroxybutyrate” or GHB, unless otherwise specified, refers to the free base of gamma-hydroxybutyrate and any pharmaceutical composition that releases free GHB base into the bloodstream of a patient, including a pharmaceutically acceptable salt of gamma-hydroxybutyric acid, a prodrug of gamma-hydroxybutyrate, their hydrates, solvates, complexes, or tautomer forms, and combinations or mixtures thereof. Gamma-hydroxybutyric acid salts may be selected from the sodium salt of gamma-hydroxybutyric acid or sodium oxybate, the potassium salt of gamma-hydroxybutyric acid, the magnesium salt of gamma-hydroxybutyric acid, the calcium salt of gamma-hydroxybutyric acid, the lithium salt of gamma-hydroxybutyric, the tetra ammonium salt of gamma-hydroxybutyric acid or any other pharmaceutically acceptable salt forms of gamma-hydroxybutyric acid.

When used herein the term “divalproex sodium” or DVP, unless otherwise specified may include divalproex sodium, divalproic acid, valproic acid, valproate, an acid or salt of valproate, or a monocarboxylate transporter.

As used herein, the term “full dose” or “full dosage” refers to the dosage amount that would be administered to the patient without co-administration. For example, a full dosage of the GHB composition refers to the dosage that would be administered to the patient without co-administration of DVP and a full dosage of DVP refers to the dosage that would be administered to the patient without co-administration with the GHB composition.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. The term “formulation” or “composition” refers to the quantitative and qualitative characteristics of a drug product or dosage form prepared in accordance with the current invention.

As used herein the doses and strengths of gamma-hydroxybutyrate are expressed in equivalent-gram (g) weights of sodium oxybate unless stated expressly to the contrary. Thus, when considering a dose of gamma-hydroxybutyrate other than the sodium salt of gamma-hydroxybutyrate, one must convert the recited dose or strength from sodium oxybate to the gamma-hydroxybutyrate under evaluation. Thus, if an embodiment is said to provide a 4.5 g dose of gamma-hydroxybutyrate, because the form of gamma-hydroxybutyrate is not specified, it will be understood that the dose encompasses a 4.5 g dose of sodium oxybate, a 5.1 g dose of potassium gamma-hydroxybutyrate (assuming a 126.09 g/mol MW for sodium oxybate and a 142.20 g/mol MW for potassium gamma-hydroxybutyrate), and a 3.7 g dose of the free base (assuming a 126.09 g/mol MW for sodium oxybate and a 104.1 g/mol MW for the free base of gamma-hydroxybutyrate), or by the weight of any mixture of salts of gamma-hydroxybutyric acid that provides the same amount of GHB as 4.5 g of sodium oxybate.

As used herein “microparticle” means any discreet particle of solid material. The particle may be made of a single material or have a complex structure with core and shells and be made of several materials. The terms “microparticle”, “particle”, “microspheres” or “pellet” are interchangeable and have the same meaning. Unless otherwise specified, the microparticle has no particular particle size or diameter and is not limited to particles with volume mean diameter D(4,3) below 1 mm.

As used herein, the “volume mean diameter D(4,3)” is calculated according to the following formula:

D(4,3)=Σ(d4i·ni)/Σ(d3i·ni)

wherein the diameter d of a given particle is the diameter of a hard sphere having the same volume as the volume of that particle.

As used herein, the terms “composition”, “oral composition”, “oral pharmaceutical composition”, “finished composition”, “finished formulation” or “formulation” are interchangeable and designate the composition of gamma-hydroxybutyrate comprising modified release microparticles of gamma-hydroxybutyrate, immediate release microparticles of gamma-hydroxybutyrate, and any other excipients. The composition may be described as extended release, delayed release, or modified release.

As used herein, “immediate release” means release of the major part of gamma-hydroxybutyrate over a relatively short period, e.g. at least 75% of the AP is released in 0.75 h, for example, in 30 min.

As used herein, an “immediate release (IR) portion” of a formulation includes physically discreet portions of a formulation, mechanistically discreet portions of a formulation, and pharmacokinetically discreet portions of a formulation that lend to or support a defined IR pharmacokinetic characteristic. Thus, for example, any formulation that releases active ingredient at the rate and extent required of the immediate release portion of the formulations of the present invention includes an “immediate release portion,” even if the immediate release portion is physically integrated in what might otherwise be considered an extended release formulation. Thus, the IR portion may be structurally discreet or structurally indiscreet from (i.e. integrated with) the MR portion. In an embodiment, the IR portion and MR portion are provided as particles, and in other embodiments the IR portion and MR portion are provided as particles discreet from each other.

As used here in, “immediate release formulation” or “immediate release portion” refers to a composition that releases at least 80% of its gamma-hydroxybutyrate in 1 hour when tested in a dissolution apparatus 2 according to USP 38 <711> in a 0.1N HCl dissolution medium at a temperature of 37° C. and a paddle speed of 75 rpm.

In like manner, a “modified-release (MR) portion” includes that portion of a formulation or dosage form that lends to or supports a particular MR pharmacokinetic characteristic, regardless of the physical formulation in which the MR portion is integrated. The modified release drug delivery systems are designed to deliver drugs at a specific time or over a period of time after administration, or at a specific location in the body. The USP defines a modified release system as one in which the time course or location of drug release or both, are chosen to accomplish objectives of therapeutic effectiveness or convenience not fulfilled by conventional IR dosage forms. More specifically, MR solid oral dosage forms include extended release (ER) and delayed-release (DR) products. A DR product is one that releases a drug all at once at a time other than promptly after administration. Typically, coatings (e.g., enteric coatings) are used to delay the release of the drug substance until the dosage form has passed through the acidic medium of the stomach. An ER product is formulated to make the drug available over an extended period after ingestion, thus allowing a reduction in dosing frequency compared to a drug presented as a conventional dosage form, e.g. a solution or an immediate release dosage form. For oral applications, the term “extended-release” is usually interchangeable with “sustained-release”, “prolonged-release” or “controlled-release”.

Traditionally, extended-release systems provided constant drug release to maintain a steady concentration of drug. For some drugs, however, zero-order delivery may not be optimal and more complex and sophisticated systems have been developed to provide multi-phase delivery. One may distinguish among four categories of oral MR delivery systems: (1) delayed-release using enteric coatings, (2) site-specific or timed release (e.g. for colonic delivery), (3) extended-release (e.g., zero-order, first-order, biphasic release, etc.), and (4), programmed release (e.g., pulsatile, delayed extended release, etc.) See Modified Oral Drug Delivery Systems at page 34 in Gibaldi's DRUG DELIVERY SYSTEMS IN PHARMACEUTICAL CARE, AMERICAN SOCIETY OF HEALTH-SYSTEM PHARMACISTS, 2007 and Rational Design of Oral Modified-release Drug Delivery Systems at page 469 in DEVELOPING SOLID ORAL DOSAGE FORMS: PHARMACEUTICAL THEORY AND PRACTICE, Academic Press, Elsevier, 2009. As used herein, “modified release formulation” or “modified release portion” in one embodiment refers to a composition that releases its gamma-hydroxybutyrate according a multiphase delivery that is comprised in the fourth class of MR products, e.g. delayed extended release. As such it differs from the delayed release products that are classified in the first class of MR products.

As used herein the terms “coating”, “coating layer,” “coating film,” “film coating” and like terms are interchangeable and have the same meaning. The terms refer to the coating applied to a particle comprising the gamma-hydroxybutyrate that controls the modified release of the gamma-hydroxybutyrate.

A “similar PK profile”, a “substantially similar PK profile”, or “comparable bioavailability” means that the mean AUC_{inf} of a test product co-administered with divalproex sodium is from 80% to 125% of the mean AUC_{inf} of same dosage of the test product administered alone in a suitably designed cross-over trial, the mean plasma concentration at 8 hours (C₈ₕ) of the test product co-administered with divalproex sodium is from 40% to 130% of the mean C₈ₕ of the reference product administered alone, and/or that the maximum plasma concentration (Cₘₐₓ) of the test product co-administered with divalproex sodium is from 50% to 140% of the Cₘₐₓ of the reference product administered alone.

As used herein, “dose proportional” occurs when increases in the administered dose are accompanied by proportional increases in the PK profile, such as the AUC or Cₘₐₓ.

A “concomitant PK profile” means the mean AUC_{inf}, the mean plasma concentration at 8 hours (C₈ₕ), and/or the maximum plasma concentration (Cₘₐₓ) of the composition when co-administered with divalproex sodium.

A “standard PK profile” means the mean AUC_{inf}, the mean plasma concentration at 8 hours (C₈ₕ), and/or the maximum plasma concentration (Cₘₐₓ) of the composition when administered alone (i.e. without co-administration with divalproex sodium).

One or more symptoms of narcolepsy include excessive daytime sleepiness (EDS), disrupted nighttime sleep (DNS), cataplexy, hypnagogic hallucinations, and sleep paralysis. Type 1 Narcolepsy (NT1) refers to narcolepsy characterized by excessive daytime sleepiness (“EDS”) and cataplexy. Type 2 Narcolepsy (NT2) refers to narcolepsy characterized by excessive daytime sleepiness without cataplexy. A diagnosis of narcolepsy (with or without cataplexy) may be confirmed by one or a combination of (i) an overnight polysomnogram (PSG) and a Multiple Sleep Latency Test (MSLT) performed within the last 2 years, (ii) a full documentary evidence confirming diagnosis from the PSG and MSLT from a sleep laboratory must be made available, (iii) current symptoms of narcolepsy including: current complaint of EDS for the last 3 months (ESS greater than 10), (iv) mean MWT less than 8 minutes, (v) mean number of cataplexy events of 8 per week on baseline Sleep/Cataplexy Diary, and/or (vi) presence of cataplexy for the last 3 months and 28 events per week during screening period.

Unless otherwise specified herein, percentages, ratios and numeric values recited herein are based on weight; averages and means are arithmetic means; all pharmacokinetic measurements based on the measurement of bodily fluids are based on plasma concentrations.

It will be understood, when defining a composition by its pharmacokinetic or dissolution properties herein, that the formulation can in the alternative be defined as “means for” achieving the recited pharmacokinetic or dissolution properties. Thus, a formulation in which the modified release portion releases less than 20% of its gamma-hydroxybutyrate at one hour can instead be defined as a formulation comprising “means for” or “modified release means for” releasing less than 20% of its gamma-hydroxybutyrate at one hour. It will be further understood that the structures for achieving the recited pharmacokinetic or dissolution properties are the structures described in the examples hereof that accomplish the recited pharmacokinetic or dissolution properties.

Oral Pharmaceutical Composition for Concomitant Administration with Divalproex Sodium

As the prior art demonstrates, it is extremely difficult to find a sodium oxybate formulation that may be concomitantly administered with divalproex sodium without reducing the dosage of sodium oxybate. It is also difficult to find a sodium oxybate formulation that when concomitantly administered with divalproex sodium has pharmacokinetic properties comparable to the sodium oxybate formulation without concomitant administration of divalproex sodium. The prior art, including the label for Xyrem, clearly teaches away from co-administering sodium oxybate and divalproex sodium at full doses. In fact, the label for Xyrem includes multiple statements recommending a reduction in the dose of Xyrem by at least 20% when co-administered with divalproex sodium based on a clinical trial finding “concomitant use of Xyrem with divalproex sodium resulted in a 25% mean increase in systemic exposure to Xyrem”.

The inventors have discovered a novel relationship between in vivo gamma-hydroxybutyrate absorption of modified release particles and the effect of divalproex sodium on the absorption of gamma-hydroxybutyrate which permits, for the first time, a full dose of a composition of gamma-hydroxybutyrate that may be concomitantly administered with divalproex sodium that approximates the bioavailability of the same composition of gamma-hydroxybutyrate at the same dose without administration of divalproex sodium, and that does so across a range of therapeutic doses. The dose of divalproex sodium administered may be a full dose that would be administered without administration of gamma-hydroxybutyrate.

Provided herein is an oral pharmaceutical composition for the treatment of narcolepsy, such as one or more symptoms of narcolepsy (e.g., excessive daytime sleepiness (EDS), disrupted nighttime sleep (DNS), cataplexy, hypnagogic hallucinations, and/or sleep paralysis) that includes gamma-hydroxybutyrate in a unit dose suitable for concomitant administration with divalproex sodium. In various embodiments, the composition may include gamma-hydroxybutyrate in an extended-release formulation, delayed release formulation, or modified release formulation.

The Xyrem® label indicates that there is a drug-drug interaction between Xyrem® and divalproex sodium, such that the divalproex sodium impacts the bioavailability of the Xyrem®, resulting in a recommendation that the Xyrem® dosage should be reduced when co-administered with divalproex sodium. In addition, the Xyrem risk evaluation and mitigation strategy (REMS) Program is a monitoring component that requires specific risk mitigation actions for the DDI between Xyrem and divalproex sodium. The FDA has concluded that information regarding the DDI with divalproex sodium cannot be “carved out” from an ANDA for a sodium oxybate product referencing Xyrem®. Based on literature data on GHB and competitive elimination pathway with divalproate, similar results as Xyrem® would have been expected. However, surprisingly, the gamma-hydroxybutyrate composition may be co-administered with divalproex sodium without being significantly impacted by the divalproex sodium. The gamma-hydroxybutyrate composition is a once daily composition with two waves of release of GHB. Without being limited to any particular theory, the two wave release of the gamma-hydroxybutyrate composition may allow for co-administration with divalproex sodium without reducing the GHB dosage. For example, the first wave may behave similarly as the reference Xyrem, while the second wave, releasing latter in the gastrointestinal tract may skip a part of the competition on the metabolic pathway, resulting in a lower interaction effect with divalproex sodium.

In an embodiment, the gamma-hydroxybutyrate composition may be co-administered with divalproex sodium without having to reduce the dosage of the gamma-hydroxybutyrate composition at any time during administration. In an embodiment, the divalproex sodium may be co-administered with the gamma-hydroxybutyrate composition without having to reduce the dosage of the divalproex sodium at any time during administration. For example, the gamma-hydroxybutyrate composition may be administered to a patient in need thereof that is already taking divalproex sodium without reducing the dosage of the gamma-hydroxybutyrate composition compared to the dosage that would be administered if the patient were not taking divalproex sodium. In another example, divalproex sodium may be administered to a patient in need thereof that is already taking the gamma-hydroxybutyrate composition without reducing the dosage of the gamma-hydroxybutyrate composition the patent is currently taking. Because the present gamma-hydroxybutyrate composition may be co-administered with divalproex sodium without reducing the dosage of either composition, there may be a reduced need for a monitoring component or no monitoring component. For example, the gamma-hydroxybutyrate composition may not need a prescriber information/brochure and/or patient counseling information relating to co-administration with divalproex sodium.

The Xyrem® label explicitly teaches that Xyrem® should not be co-administered with divalproex sodium without reducing the dosage of Xyrem® by 20%, as the divalproex sodium increases the systemic exposure of gamma-hydroxybutyrate from Xyrem beyond 25% of systemic exposure when Xyrem® is administered alone. Contrary to this, concomitant use of the present gamma-hydroxybutyrate composition with divalproex sodium may result in a lower change in systemic exposure to the gamma-hydroxybutyrate composition, as compared to concomitant administration of Xyrem® and divalproex sodium. For example, concomitant use of the gamma-hydroxybutyrate composition with divalproex sodium may result in a less than 25% mean increase in systemic exposure to the gamma-hydroxybutyrate composition. In some examples, concomitant use of the gamma-hydroxybutyrate composition with divalproex sodium may result in a less than 15% mean increase in systemic exposure to the gamma-hydroxybutyrate composition. In other examples, concomitant use of the gamma-hydroxybutyrate composition with divalproex sodium may result in a less than 5% mean increase in systemic exposure to the gamma-hydroxybutyrate composition. In at least one example, concomitant use of the gamma-hydroxybutyrate composition with divalproex sodium may result in no change in systemic exposure to the gamma-hydroxybutyrate composition.

The Xyrem® label also explicitly teaches that Xyrem® co-administered with divalproex sodium can result in impairment on some tests of attention and working memory. Surprisingly, concomitant use of the gamma-hydroxybutyrate composition with divalproex sodium may result in fewer side effects, as compared to concomitant administration of Xyrem® and divalproex sodium. For example, concomitant use of the gamma-hydroxybutyrate composition with divalproex sodium may result in less impairment on some tests of attention and working memory, as compared to concomitant administration of Xyrem® and divalproex sodium. In other examples, patients may not reduce the dosage without risking side effects of GHB overdosage.

The oral pharmaceutical composition of gamma-hydroxybutyrate may be in a unit dose suitable for co-administration with divalproex sodium without reducing the dosage of gamma-hydroxybutyrate for the treatment of narcolepsy or one or more symptoms of narcolepsy (e.g., one or more symptoms of narcolepsy selected from excessive daytime sleepiness (EDS), disrupted nighttime sleep (DNS), cataplexy, hypnagogic hallucinations, and sleep paralysis) in a human subject in need thereof. In some embodiments, the oral pharmaceutical composition may be effective to treat narcolepsy, cataplexy, or excessive daytime sleepiness in a human subject in need thereof. In some examples, the human subject may be a human patient. In any of the embodiments provided herein, the formulation may be effective to treat narcolepsy Type 1 or Type 2. The treatment of narcolepsy may be defined as reducing excessive daytime sleepiness, reducing the frequency of cataplectic attacks, reducing disrupted nighttime sleep, reducing hypnagogic hallucinations, or reducing sleep paralysis. In various embodiments, the composition is sufficient to be administered once daily. For example, the composition may be sufficient to administer in the morning or at night concomitant with divalproex sodium. The formulation is also effective to induce sleep for at least 6 to 8 consecutive hours. In one embodiment, the composition co-administered with divalproex sodium is effective to induce sleep for at least 8 consecutive hours. In various embodiments, the formulation is effective to induce sleep for at least 6 hours, at least 7 hours, at least 8 hours, at least 9 hours, or at least 10 hours. In other embodiments, the formulation is effective to induce sleep for up to 6 hours, up to 7 hours, up to 8 hours, up to 9 hours, or up to 10 hours.

The compositions of gamma-hydroxybutyrate may have both immediate release and modified release portions. The release of gamma-hydroxybutyrate from the immediate release portion is practically uninhibited, and occurs almost immediately in 0.1N hydrochloric acid dissolution medium. In contrast, while the modified release portion also may release its gamma-hydroxybutyrate almost immediately when fully triggered, the release is not triggered until a predetermined lag-time or the drug is subjected to a suitable dissolution medium such as a phosphate buffer pH 6.8 dissolution medium. Without wishing to be bound by any theory, it is believed that divalproex sodium may have no or low impact on the modified release portion of the composition, as the gamma-hydroxybutyrate from the modified release portion is absorbed in the latter part of the gastro-intestinal tract.

In any of these embodiments, the composition may include immediate release and modified release portions, where the modified release portion includes gamma hydroxybutyrate particles coated by a polymer carrying free carboxylic groups and a hydrophobic compound having a melting point equal or greater than 40° C., and the ratio of gamma-hydroxybutyrate in the immediate release portion and the modified release portion is from 10/90 to 65/35. The polymers comprising free carboxylic groups may have a pH dissolution trigger of from 5.5 to 6.97 and may be methacrylic acid copolymers having a pH dissolution trigger of from 5.5 to 6.97.

In various embodiments, the composition includes gamma-hydroxybutyrate present in a unit dose of at least 4.5 g, at least 6.0 g, at least 7.5 g, or at least 9.0 g. In some embodiments, the oral pharmaceutical composition of gamma-hydroxybutyrate may be administered as a once-daily dose concomitantly with a dose of divalproex sodium. In an example, the once-daily dose of the gamma-hydroxybutyrate is administered as a 6 g dose. The once-daily dose of the gamma-hydroxybutyrate may be administered once nightly. In an example, the once-nightly dose of the gamma-hydroxybutyrate is administered as a 6 g dose. The dose range of divalproex sodium ER is 10 to 60 mg/kg body weight per day. In some examples, the divalproex sodium is administered up to a daily dose of 60 mg/kg/day. In other examples, the divalproex sodium is administered at a dose of 1250 mg/day.

### Pharmacokinetics

The composition may provide a substantially similar concomitant PK profile and standard PK profile when the gamma-hydroxybutyrate composition is administered at the same dose. In some embodiments, the concomitant administration of gamma-hydroxybutyrate and divalproex sodium provides a substantially bioequivalent PK profile as compared to administration of an equal dose of the gamma-hydroxybutyrate composition in the absence of the concomitant administration of divalproex sodium.

In an embodiment, compositions of gamma-hydroxybutyrate co-administered with divalproex sodium may roughly approximate the bioavailability of an equal dose of the gamma-hydroxybutyrate composition without divalproex sodium, across the entire therapeutic range of gamma-hydroxybutyrate doses.

In an embodiment, there is no significant reduction in safety or efficacy to a patient following co-administration of the composition with divalproex sodium. For example, the safety profile for co-administration of the gamma-hydroxybutyrate composition and divalproex sodium may be consistent with what is known for sodium oxybate.

In another embodiment, the compositions of gamma-hydroxybutyrate may allow co-administration with divalproex sodium without a reduction in the dosage of gamma-hydroxybutyrate as compared to the commercial treatment Xyrem® which requires a reduction in the Xyrem® dosage by at least 5% when co-administered with divalproex sodium. In some examples, the dosage of the gamma-hydroxybutyrate composition is reduced by less than 5% in response to the concomitant administration of DVP.

In another embodiment, divalproex sodium may be co-administered with the compositions of gamma-hydroxybutyrate without a reduction in the dosage of divalproex sodium.

In other embodiments, the compositions of gamma-hydroxybutyrate may be co-administered with divalproex with improved dissolution and pharmacokinetic profiles compared to co-administration of Xyrem® and divalproex without reducing the Xyrem® dosage.

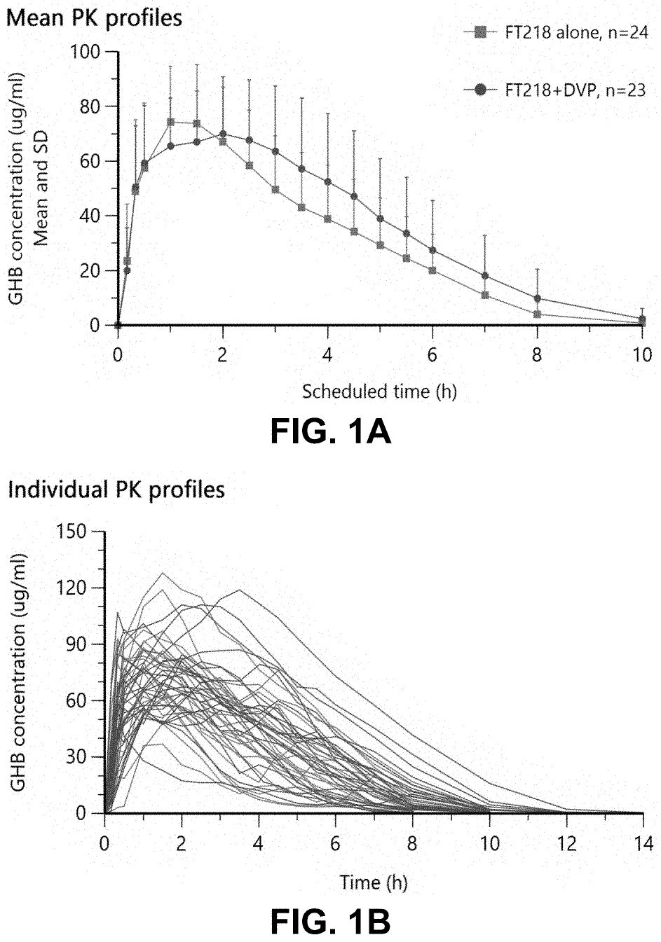

The compositions of gamma-hydroxybutyrate may also be defined by the concentration/time curves that they produce when tested according to the Examples. An embodiment of the composition of gamma-hydroxybutyrate yields a plasma concentration versus time curve when administered at a strength of 6 g concomitantly with divalproex sodium substantially as depicted in FIGS. 1A and 1B.

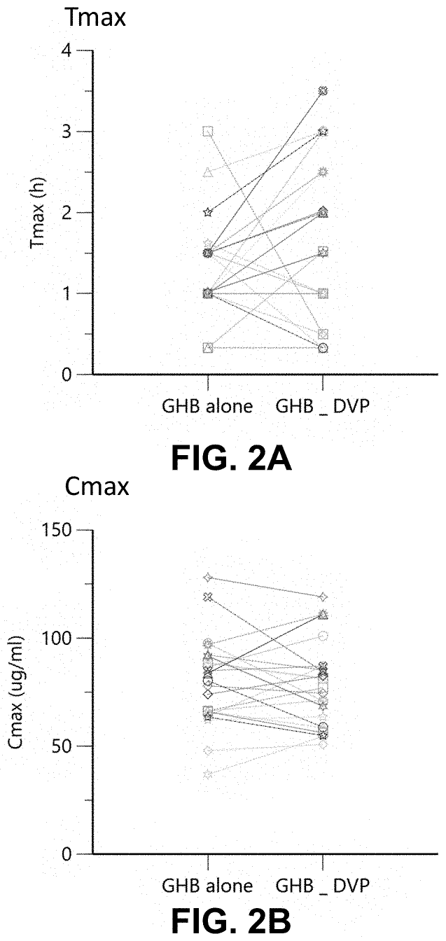

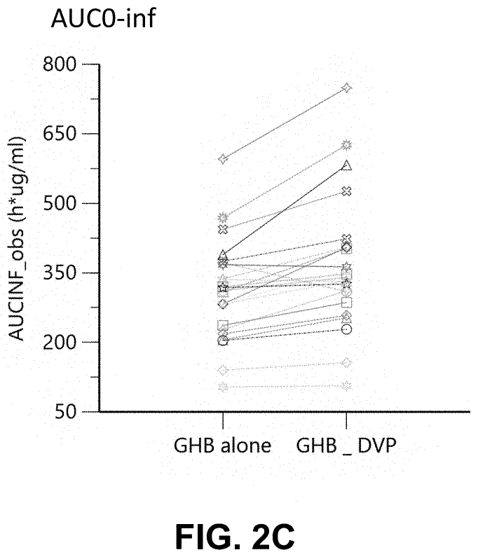

In an embodiment, concomitant administration of the gamma-hydroxybutyrate composition and divalproex sodium provides a Tₘₐₓ bioequivalent to a Tₘₐₓ of the same dosage of the gamma-hydroxybutyrate composition alone, as depicted in FIG. 2A. In another embodiment, concomitant administration of the gamma-hydroxybutyrate composition and divalproex sodium provides a Cₘₐₓ bioequivalent to a Cₘₐₓ of the same dosage of the gamma-hydroxybutyrate composition alone, as depicted in FIG. 2B. In an embodiment, concomitant administration of the gamma-hydroxybutyrate composition and divalproex sodium provides an AUC_{inf} bioequivalent to an AUC_{inf} of the same dosage of the gamma-hydroxybutyrate composition alone, as depicted in FIG. 2C.

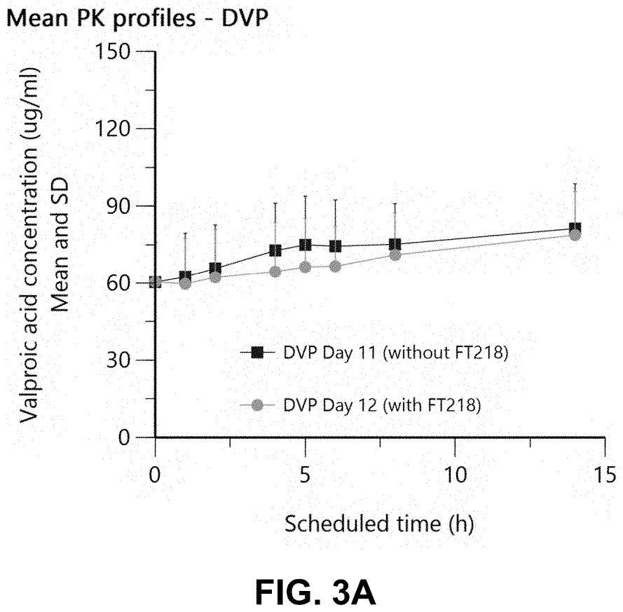

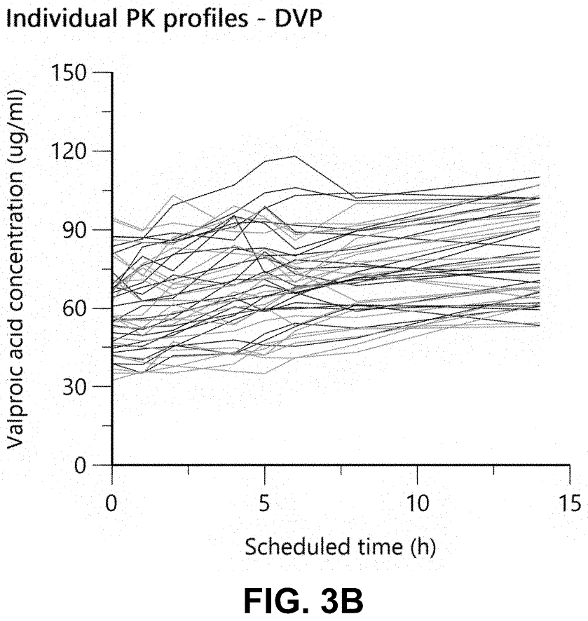

In yet another embodiment, divalproex sodium yields a plasma concentration versus time curve when co-administered with the composition of gamma-hydroxybutyrate once nightly at a strength of 6 g substantially as depicted in FIGS. 3A and 3B.

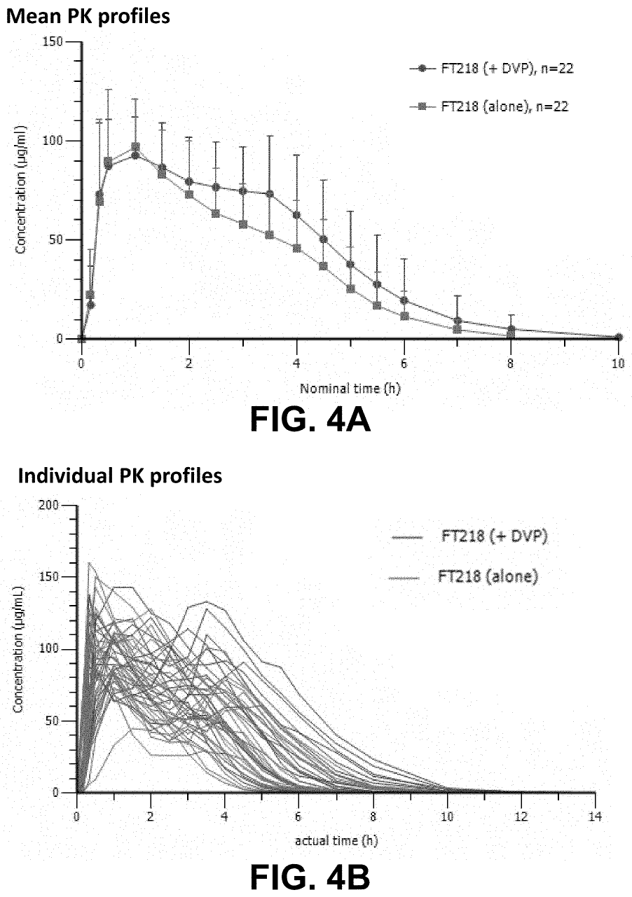

Another embodiment of the composition of gamma-hydroxybutyrate yields a plasma concentration versus time curve when administered once nightly at a strength of 6 g concomitantly with divalproex sodium substantially as depicted in FIG. 4.

Formulations that achieve this improved bioavailability when co-administered with divalproex sodium may be described using several different pharmacokinetic parameters. Compositions of gamma-hydroxybutyrate administered once nightly concomitantly with divalproex sodium may achieve a relative bioavailability of greater than 80%, 85%, 90%, or 95% when compared to an equal dose of the gamma-hydroxybutyrate composition administered without divalproex sodium.

In an embodiment, the AUC_{inf} for the gamma-hydroxybutyrate composition administered concomitantly with divalproex sodium may be substantially similar to the AUC_{inf} when the same the same dosage of the composition is administered alone. For example, when the gamma-hydroxybutyrate composition is co-administered with divalproex sodium, it achieves a mean AUC_{inf} that is from 80% to 125%, from 80% to 100%, from 90% to 100%, from 90% to 115%, from 100% to 120%, or from 110% to 125% of the mean AUC_{inf} provided by an equal dose of the gamma-hydroxybutyrate composition administered without divalproex sodium. In at least one example, when the gamma-hydroxybutyrate composition is co-administered with divalproex sodium, it achieves a mean AUC_{inf} that is about 117% of the mean AUC_{inf} provided by an equal dose of the gamma-hydroxybutyrate composition administered without divalproex sodium. This may be seen by comparing the release profiles and pharmacokinetic profiles in Examples 1-6.

An embodiment of the composition of gamma-hydroxybutyrate includes immediate release and modified release portions, where a 6 g dose of the formulation, when administered with divalproex sodium, may achieve a mean AUC_{inf} of greater than 220 hr*μg/mL. In particular, a 6 g dose of a composition of gamma-hydroxybutyrate co-administered with divalproex may achieve a mean AUC_{inf} of greater than 250 hr*μg/mL, 300 hr*μg/mL, 350 hr*μg/mL, 400 hr*μg/mL, 450 hr*μg/mL, 500 hr*μg/mL, or less than 512 hr*μg/mL. For example, a 6 g dose of the composition co-administered with divalproex sodium may have a mean AUC_{inf} of about 366 hr*μg/mL. In addition, the 6 g dose of the composition may be administered once daily, in the morning or the evening.

The AUC_{inf} for the composition administered with DVP is within the bioequivalent range of the same composition administered alone. In various examples, the 90% confidence interval of the geometric mean ratio of the AUC_{inf} for the composition and DVP/composition (alone) is about 111 to about 122. In at least one example, the ratio is about 116.52.

In an embodiment, the Cₘₐₓ for the gamma-hydroxybutyrate composition administered concomitantly with divalproex sodium may be substantially similar to the Cₘₐₓ when the same dosage of the gamma-hydroxybutyrate composition is administered alone. In an example, when the gamma-hydroxybutyrate composition is co-administered with divalproex sodium, it achieves a mean Cₘₐₓ that is from 80% to 125%, from 80% to 100%, from 90% to 100%, from 95% to 110%, from 100% to 120%, or from 110% to 125% of the mean Cₘₐₓ provided by an equal dose of the gamma-hydroxybutyrate composition administered without divalproex sodium. In at least one example, when the gamma-hydroxybutyrate composition is co-administered with divalproex sodium, it achieves a mean Cₘₐₓ that is about 98% of the mean Cₘₐₓ provided by an equal dose of the gamma-hydroxybutyrate composition administered without divalproex sodium. This may be seen by comparing the release profiles and pharmacokinetic profiles in Examples 1-6.

An embodiment of the composition of gamma-hydroxybutyrate includes immediate release and modified release portions, where a 6 g dose of the formulation, when administered with divalproex sodium, may achieve a mean Cₘₐₓ of greater than 59 μg/mL. For example, a 6 g dose of the formulation, when co-administered with divalproex sodium, may achieve a mean Cₘₐₓ of greater than 65 μg/mL, 70 μg/mL, 75 μg/mL, 80 μg/mL, 85 μg/mL, 90 μg/mL, 95 μg/mL, or less than 97 μg/mL. For example, a 6 g dose of the composition co-administered with divalproex sodium has a mean Cₘₐₓ of about 78 μg/mL. In addition, the 6 g dose of the composition may be administered once daily, in the morning or the evening.

The Cₘₐₓ for the composition administered with DVP is within the bioequivalent range of the same composition administered alone. In various examples, the 90% confidence interval of the geometric mean ratio of the Cₘₐₓ for the composition and DVP/composition (alone) is about 91 to about 106. In at least one example, the ratio is about 98.46.

In an embodiment, the AUC₀₋ₗₐₛₜ for the gamma-hydroxybutyrate composition administered concomitantly with divalproex sodium may be substantially similar to the AUC₀₋ₗₐₛₜ when the same the same dosage of the gamma-hydroxybutyrate composition is administered alone. In some examples, when the gamma-hydroxybutyrate composition is co-administered with divalproex sodium, it achieves a mean AUC₀₋ₗₐₛₜ that is from 80% to 125%, from 80% to 100%, from 90% to 100%, from 95% to 110%, or from 100% to 125% of the mean AUC₀₋ₗₐₛₜ provided by an equal dose of the gamma-hydroxybutyrate composition administered without divalproex sodium. In at least one example, when the gamma-hydroxybutyrate composition is co-administered with divalproex sodium, it achieves a mean AUC₀₋ₗₐₛₜ that is about 117% of the mean AUC₀₋ₗₐₛₜ provided by an equal dose of the gamma-hydroxybutyrate composition administered without divalproex sodium. This may be seen by comparing the release profiles and pharmacokinetic profiles in Examples 1-6.

In various embodiments, a 6 g dose of the composition of gamma-hydroxybutyrate may be characterized as having been shown to achieve a mean AUC₀₋ₗₐₛₜ of greater than 220 hr*μg/mL, 250 hr*μg/mL, 300 hr*μg/mL, 350 hr*μg/mL, 400 hr*μg/mL, 450 hr*μg/mL, 500 hr*μg/mL, or less than 512 hr*μg/mL when co-administered with divalproex sodium. For example, a 6 g dose of the composition co-administered with divalproex sodium may have a mean AUC₀₋ₗₐₛₜ of about 366 hr*μg/mL. In addition, the 6 g dose of the composition may be administered once daily, in the morning or the evening.

The AUC₀₋ₗₐₛₜ for the composition administered with DVP is within the bioequivalent range of the same composition administered alone. In various examples, the 90% confidence interval of the geometric mean ratio of the AUC₀₋ₗₐₛₜ for the composition and DVP/composition (alone) is about 111 to about 122. In at least one example, the ratio is about 116.67.

In an embodiment, the gamma-hydroxybutyrate composition administered concomitantly with divalproex sodium may provide mean blood concentrations (μg/ml) at 8 hours substantially similar to that of the same dosage of the gamma-hydroxybutyrate composition when administered alone. In an example, when the gamma-hydroxybutyrate composition is co-administered with divalproex sodium, it achieves a mean C₈ₕ that is from 40% to 60%, from 60% to 80%, from 80% to 125%, from 80% to 100%, from 90% to 100%, from 90% to 115%, or from 100% to 125% of the mean C₈ₕ provided by an equal dose of the gamma-hydroxybutyrate composition administered without divalproex sodium. This may be seen by comparing the release profiles and pharmacokinetic profiles in Examples 1-6.

In various embodiments, a 6 g dose of the composition of gamma-hydroxybutyrate may be characterized as having been shown to achieve a mean C₈ₕ of greater than 1 μg/mL, 2 μg/mL, 4 μg/mL, 6 μg/mL, 8 μg/mL, 10 μg/mL, 12 μg/mL, 14 μg/mL, 16 μg/mL, 18 μg/mL, or 20 μg/mL when co-administered with divalproex sodium. For example, a 6 g dose of the composition co-administered with divalproex sodium has a mean C₈ₕ of about 9.8 μg/mL. In addition, the 6 g dose of the composition may be administered once daily, in the morning or the evening.

In an embodiment, the Tₘₐₓ for the gamma-hydroxybutyrate composition administered concomitantly with divalproex sodium may be substantially similar to the Tₘₐₓ when the same dosage of the gamma-hydroxybutyrate composition is administered alone. In some examples, when the gamma-hydroxybutyrate composition is co-administered with divalproex sodium, it achieves a mean Tₘₐₓ that is from 60% to 80%, from 70% to 90%, 80% to 125%, from 80% to 100%, from 90% to 100%, from 90% to 115%, or from 100% to 125% of the mean Tₘₐₓ provided by an equal dose of the gamma-hydroxybutyrate composition administered without divalproex sodium. This may be seen by comparing the release profiles and pharmacokinetic profiles in Examples 1-6.

The compositions of gamma-hydroxybutyrate may also be defined based on the time required to reach maximum blood concentration of gamma-hydroxybutyrate. Thus, in additional embodiments, the composition of gamma-hydroxybutyrate may achieve a mean Tₘₐₓ of 0.3 to 3.5 hours. In various embodiments, the composition of gamma-hydroxybutyrate may achieve a mean Tₘₐₓ of about 0.5, 0.75 hours, 1.0 hour, 1.5 hours, 2.0 hours, 2.25 hours, 2.5 hours, 3 hours, or 3.5 hours when co-administered with divalproex sodium. For example, a 6 g dose of the composition co-administered with divalproex sodium may have a median Tₘₐₓ of about 2 hours. In addition, the 6 g dose of the composition may be administered once daily, in the morning or the evening.

In an embodiment, the composition provides an AUC_{inf} that is dose proportional when co-administered with divalproex sodium. In an embodiment, the composition provides a Cₘₐₓ that is dose proportional when co-administered with divalproex sodium. In various embodiments, the composition exhibits pharmacokinetics that is dose proportional when administered once daily, concomitant with divalproex sodium. For example, the composition provides a Cₘₐₓ that is dose proportional across once daily doses of 4.5 g, 7.5 g, 6 g, and 9 g, such that, the Cₘₐₓ of a 6 g dose is proportional to the Cₘₐₓ of a 9 g dose of the composition. The composition may exhibit predictable increases in plasma levels with increasing doses, consistent with the PK profile desired for a once-nightly sodium oxybate formulation.

### Structural Embodiments

The compositions of gamma-hydroxybutyrate may be provided in any dosage form that is suitable for oral administration, including tablets, capsules, liquids, orally dissolving tablets, and the like. In one embodiment, they are provided as dry particulate formulations (i.e. granules, powders, coated particles, microparticles, pellets, microspheres, etc.), in a sachet or other suitable discreet packaging units. A particulate formulation will be mixed with tap water shortly before administration. In one embodiment, the composition may be mixed with 50 mL water prior to administration. In another embodiment, the composition is an oral pharmaceutical composition.

In various embodiments, the composition includes gamma-hydroxybutyrate present in a unit dose of at least 4.5 g, at least 6.0 g, at least 7.5 g, or at least 9.0 g. In various embodiments, the composition includes gamma-hydroxybutyrate present in a unit dose of more than 4.5 g, more than 6.0 g, more than 7.5 g, or more than 9.0 g. In one example, the formulation includes 6 g gamma-hydroxybutyrate. In another example, the formulation includes 7.5 g gamma-hydroxybutyrate. In yet another example, the formulation includes 9 g gamma-hydroxybutyrate. In some embodiments, the dosage of gamma-hydroxybutyrate may be sufficient to administer the composition once daily.

In one embodiment, the formulation comprises immediate release and modified release portions, wherein: (a) the modified release portion comprises coated microparticles of gamma-hydroxybutyrate; and (b) the ratio of gamma-hydroxybutyrate in the immediate release portion and the modified release portion is from 10/90 to 65/35.

In one embodiment, the formulation comprises immediate release and modified release portions, wherein: (a) the modified release portion comprises coated microparticles of gamma-hydroxybutyrate; and (b) the ratio of gamma-hydroxybutyrate in the immediate release portion and the modified release portion is from 40/60 to 60/40.

In another embodiment, the formulation comprises immediate release and modified release portions, wherein: (a) the modified release portion comprises coated microparticles of gamma-hydroxybutyrate; (b) the coating of said modified release particles of gamma-hydroxybutyrate comprises a polymer carrying free carboxylic groups and a hydrophobic compound having a melting point equal or greater than 40° C.; and (c) the ratio of gamma-hydroxybutyrate in the immediate release portion and the modified release portion is from 10/90 to 65/35 or 40/60 to 60/40.

In an embodiment, the composition of gamma-hydroxybutyrate may include immediate release and modified release portions, a suspending or viscosifying agent, and an acidifying agent, wherein: (a) the modified release portion comprises coated particles of gamma-hydroxybutyrate; (b) the coating comprises a polymer carrying free carboxylic groups and a hydrophobic compound having a melting point equal or greater than 40° C.; and (c) the ratio of gamma-hydroxybutyrate in the immediate release portion and the modified release portion is from 10/90 to 65/35.

In another embodiment, the formulation comprises immediate release and modified release portions, wherein: (a) the modified release portion comprises coated microparticles of gamma-hydroxybutyrate; (b) the coating of said modified release particles of gamma-hydroxybutyrate comprises a polymer carrying free carboxylic groups and a hydrophobic compound having a melting point equal or greater than 40° C.; (c) the weight ratio of the hydrophobic compound to the polymer carrying free carboxylic groups is from 0.4 to 4; (d) the ratio of gamma-hydroxybutyrate in the immediate release portion and the modified release portion is from 10/90 to 65/35 or 40/60 to 60/40; and (e) the film coating is from 10 to 50% of the weight of the microparticles.

In another embodiment the formulation comprises immediate release and modified release portions, wherein: (a) the modified release portion comprises coated particles of gamma-hydroxybutyrate; (b) the coating of said modified release particles of gamma-hydroxybutyrate comprises a polymer carrying free carboxylic groups having a pH trigger of from 5.5 to 6.97 and a hydrophobic compound having a melting point equal or greater than 40° C.; (c) the weight ratio of the hydrophobic compound to the polymer carrying free carboxylic groups is from 0.4 to 4; (d) the ratio of gamma-hydroxybutyrate in the immediate release portion and the modified release portion is from 10/90 to 65/35 or 40/60 to 60/40; and (e) the coating is from 10 to 50% of the weight of the particles.

In an embodiment, the polymer carrying free carboxylic groups comprises from 100% poly (methacrylic acid, ethyl acrylate) 1:1 and 0% poly (methacrylic acid, methylmethacrylate) 1:2 to 2% poly (methacrylic acid, ethyl acrylate) 1:1 and 98% poly (methacrylic acid, methylmethacrylate) 1:2; and the hydrophobic compound comprises hydrogenated vegetable oil.

In an embodiment, the formulation includes excipients to improve the viscosity and the pourability of the mixture of the particulate formulation with tap water. As such, the particulate formulation comprises, besides the immediate release and modified release particles of gamma-hydroxybutyrate, one or more suspending or viscosifying agents or lubricants.

Suspending or viscosifying agents may be chosen from the group consisting of xanthan gum, medium viscosity sodium carboxymethyl cellulose, mixtures of microcrystalline cellulose and sodium carboxymethyl cellulose, mixtures of microcrystalline cellulose and guar gum, medium viscosity hydroxyethyl cellulose, agar, sodium alginate, mixtures of sodium alginate and calcium alginate, gellan gum, carrageenan gum grade iota, kappa or lambda, and medium viscosity hydroxypropylmethyl cellulose.

Medium viscosity sodium carboxymethyl cellulose corresponds to grade of sodium carboxymethyl cellulose whose viscosity, for a 2% solution in water at 25° C., is greater than 200 mPa·s and lower than 3100 mPa·s.

Medium viscosity hydroxyethyl cellulose corresponds to a grade of hydroxyethyl cellulose whose viscosity, for a 2% solution in water at 25° C., is greater than 250 mPa·s and lower than 6500 mPa·s. Medium viscosity hydroxypropylmethyl cellulose corresponds to a grade of hydroxypropylmethyl cellulose whose viscosity, for a 2% solution in water at 20° C., is greater than 80 mPa·s. and lower than 3800 mPa·s.

In one embodiment, the suspending or viscosifying agents are xanthan gum, especially Xantural 75™ from Kelco, hydroxyethylcellulose, especially Natrosol 250M™ from Ashland, Kappa carrageenan gum, especially Gelcarin PH812™ from FMC Biopolymer, and lambda carrageenan gum, especially Viscarin PH209™ from FMC Biopolymer.

In an embodiment, the composition of gamma-hydroxybutyrate comprises from 1 to 15% of viscosifying or suspending agents. In other embodiments, the composition of gamma-hydroxybutyrate comprises viscosifying or suspending agents in an amount from 2 to 10%, from 2 to 5%, or from 2 to 3% of the formulation.

In an embodiment, the composition of gamma-hydroxybutyrate is in the form of a powder that is intended to be dispersed in water prior to administration and further comprises from 1 to 15% of a suspending or viscosifying agent selected from a mixture of xanthan gum, carrageenan gum and hydroxyethylcellulose or xanthan gum and carrageenan gum.

In an embodiment, the composition of gamma-hydroxybutyrate is in the form of a powder that is intended to be dispersed in water prior to administration and further comprises: from 1.2 to 15% of an acidifying agent selected from malic acid and tartaric acid; and from 1 to 15% of a suspending or viscosifying agent selected from a mixture of xanthan gum, carrageenan gum and hydroxyethylcellulose or xanthan gum and carrageenan gum.

In one embodiment, the composition of gamma-hydroxybutyrate comprises about 1% of lambda carrageenan gum or Viscarin PH209™, about 1% of medium viscosity grade of hydroxyethyl cellulose or Natrosol 250M™, and about 0.7% of xanthan gum or Xantural 75™. For a 4.5 g dose unit, these percentages will typically equate to about 50 mg xanthan gum (Xantural 75™), about 75 mg carragenan gum (Viscarin PH209™), and about 75 mg hydroxyethylcellulose (Natrasol 250M™).

Alternative packages of viscosifying or suspending agents, for a 4.5 g dose, include about 50 mg xanthan gum (Xantural 75™) and about 100 mg carragenan gum (Gelcarin PH812™), or about 50 mg xanthan gum (Xantural 75™), about 75 mg hydroxyethylcellulose (Natrasol 250M™), and about 75 mg carragenan gum (Viscarin PH109™).

In an embodiment, the composition of gamma-hydroxybutyrate further comprises a lubricant or a glidant, besides the immediate release and modified release particles of gamma-hydroxybutyrate. In various embodiments, the lubricants and glidants are chosen from the group consisting of salts of stearic acid, in particular magnesium stearate, calcium stearate or zinc stearate, esters of stearic acid, in particular glyceryl monostearate or glyceryl palmitostearate, stearic acid, glycerol behenate, sodium stearyl fumarate, talc, and colloidal silicon dioxide. In one embodiment, the lubricant or glidant is magnesium stearate. The lubricant or glidant may be used in the particulate formulation in an amount of from 0.1 to 5%. In one embodiment, the amount of lubricant or glidant is about 0.5%. For example, the composition of gamma-hydroxybutyrate may include about 0.5% of magnesium stearate.

A composition of gamma-hydroxybutyrate may further include an acidifying agent. The acidifying agent helps to ensure that the release profile of the formulation in 0.1N HCl will remain substantially unchanged for at least 15 minutes after mixing, which is approximately the maximum length of time a patient might require before consuming the dose after mixing the formulation with tap water.

In one embodiment, the formulation is a powder, and further comprising an acidifying agent and a suspending or viscosifying agent in the weight percentages recited herein.

The acidifying agents may be chosen from the group consisting of malic acid, citric acid, tartaric acid, adipic acid, boric acid, maleic acid, phosphoric acid, ascorbic acid, oleic acid, capric acid, caprylic acid, and benzoic acid. In various embodiments, the acidifying agent is present in the formulation from 1.2 to 15%, from 1.2 to 10%, or from 1.2 to 5%. In one embodiment, the acidifying agents are tartaric acid and malic acid. In another embodiment, the acidifying agent is malic acid.

When tartaric acid is employed, it may be employed in an amount of from 1 to 10%, from 2.5 to 7.5%, or about 5%. In various embodiments, the amount of malic acid in the composition of gamma-hydroxybutyrate is from 1.2 to 15%, from 1.2 to 10%, from 1.2 to 5%, or from 1.6% or 3.2%. In one embodiment, the amount of malic acid in the composition of gamma hydroxybutyrate is about 1.6%.

The composition of gamma-hydroxybutyrate includes an immediate release portion and a modified release portion of gamma-hydroxybutyrate, and in an embodiment, the formulation is a particulate formulation that includes a plurality of immediate release gamma-hydroxybutyrate particles and a plurality of modified release gamma-hydroxybutyrate particles. The molar ratio of gamma-hydroxybutyrate in the immediate release and modified release portions ranges from 0.11:1 to 1.86:1, from 0.17:1 to 1.5:1, from 0.25:1 to 1.22:1, from 0.33:1 to 1.22:1, from 0.42:1 to 1.22:1, from 0.53:1 to 1.22:1, from 0.66:1 to 1.22:1, from 0.66:1 to 1.5:1, from 0.8:1 to 1.22:1. In one embodiment, the molar ratio of gamma-hydroxybutyrate in the immediate release and modified release portions is about 1:1. The molar percentage of gamma-hydroxybutyrate in the immediate release portion relative to the total of gamma-hydroxybutyrate in the formulation ranges from 10% to 65%, from 15 to 60%, from 20 to 55%, from 25 to 55%, from 30 to 55%, from 35 to 55%, from 40 to 55%, from 40 to 60%, or from 45 to 55%. In one embodiment, the molar percentage of gamma-hydroxybutyrate in the immediate release portion relative to the total of gamma-hydroxybutyrate in the formulation ranges from 40% to 60%. In an embodiment, the molar percentage of the gamma-hydroxybutyrate in the immediate release portion relative to the total of gamma-hydroxybutyrate in the formulation is about 50%. The molar percentage of gamma-hydroxybutyrate in the modified release portion relative to the total of gamma-hydroxybutyrate in the formulation ranges from 90% to 35%, from 85 to 40%, from 80 to 45%, from 75 to 45%, from 70 to 45%, from 65 to 45%, from 60 to 45%, from 60 to 40%, or from 55 to 45%. In an embodiment, the molar percentage of gamma-hydroxybutyrate in the modified release portion relative to the total of gamma-hydroxybutyrate in the formulation ranges from 60% to 40%. In one embodiment, the molar ratio of the gamma-hydroxybutyrate in the modified release portion relative to the total of gamma-hydroxybutyrate in the formulation is about 50%. The weight percentage of the IR microparticles relative to the total weight of IR microparticles and MR microparticles ranges from 7.2% to 58.2%, from 11.0% to 52.9%, from 14.9% to 47.8%, from 18.9% to 47.8%, from 23.1% to 47.8%, from 27.4% to 47.8%, from 31.8% to 47.8%, from 31.8% to 52.9%, or from 36.4% to 47.8%. In other embodiments, the weight percentage of the IR microparticles relative to the total weight of IR microparticles and MR microparticles ranges from 5.9% to 63.2%, from 9.1% to 58.1%, from 12.4% to 53.1%, from 19.9% to 53.1%, from 19.6% to 53.1%, from 23.4% to 53.1%, from 27.4% to 53.1%, or from 27.4% to 58.1%. In one embodiment, the weight percentage of the IR microparticles relative to the total weight of IR microparticles and MR microparticles ranges from 31.7% to 53.1%.

In an embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to 450 microns and 50% of its sodium oxybate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to 170 microns and 50% of its sodium oxybate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to about 450 microns and 50% of its sodium oxybate content in modified release particles consisting of 11.3% w/w of microcrystalline cellulose spheres with a volume mean diameter of about 95 microns to about 450 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

In an embodiment, the finished formulation comprises 50% of its sodium oxybate content in immediate-release particles consisting of 80.75% w/w of sodium oxybate, 4.25% w/w of Povidone™ K30 and 15% of microcrystalline cellulose spheres with a volume mean diameter of about 95 microns to about 170 microns and 50% of its sodium oxybate content in modified release particles consisting of 11.3% w/w of microcrystalline cellulose spheres with a volume mean diameter of about 95 microns to about 170 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 16.7% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns, 16.7% of its gamma-hydroxybutyrate content in immediate-release particles consisting of 80.75% w/w of magnesium salt of gamma-hydroxybutyric acid, 4.25% w/w of Povidone K30 and 15% of microcrystalline cellulose spheres with a volume mean diameter of about 95 microns to about 450 microns, 16.7% of its gamma-hydroxybutyrate content in immediate-release particles consisting of 80.75% w/w of calcium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 16.7% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns, 16.7% of its gamma-hydroxybutyrate content in immediate-release particles consisting of 80.75% w/w of magnesium salt of gamma-hydroxybutyric acid, 4.25% w/w of Povidone K30 and 15% of microcrystalline cellulose spheres with a volume mean diameter of about 95 microns to about 170 microns, 16.7% of its gamma-hydroxybutyrate content in immediate-release particles consisting of 80.75% w/w of calcium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of calcium salt of gamma-hydroxybutyric acid mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of calcium salt of gamma-hydroxybutyric acid mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

### Other Characteristics of Immediate Release Portion

The immediate release portion of the formulation can take any form capable of achieving an immediate release of the gamma-hydroxybutyrate when ingested. For example, when the formulation is a particulate formulation, the formulation can include unmodified “raw” gamma-hydroxybutyrate, rapidly dissolving gamma-hydroxybutyrate granules, particles or microparticles comprised of a core covered by a gamma-hydroxybutyrate loaded layer containing a binder such as povidone.

The IR granules or particles of gamma-hydroxybutyrate may be made using any manufacturing process suitable to produce the required particles, including:
agglomeration of the gamma-hydroxybutyrate sprayed in the molten state, such as the Glatt ProCell™ technique,
extrusion and spheronization of the gamma-hydroxybutyrate, optionally with one or more physiologically acceptable excipients,
wet granulation of the gamma-hydroxybutyrate, optionally with one or more physiologically acceptable excipients,
compacting of the gamma-hydroxybutyrate, optionally with one or more physiologically acceptable excipients,
granulation and spheronization of the gamma-hydroxybutyrate, optionally with one or more physiologically acceptable excipients, the spheronization being carried out for example in a fluidized bed apparatus equipped with a rotor, in particular using the Glatt CPS™ technique,
spraying of the gamma-hydroxybutyrate, optionally with one or more physiologically acceptable excipients, for example in a fluidized bed type apparatus equipped with zig-zag filter, in particular using the Glatt MicroPx™ technique, or
spraying, for example in a fluidized bed apparatus optionally equipped with a partition tube or Wurster tube, the gamma-hydroxybutyrate, optionally with one or more physiologically acceptable excipients, in dispersion or in solution in an aqueous or organic solvent on a core.

The immediate release portion of the formulation is in the form of microparticles comprising the immediate release gamma-hydroxybutyrate and optional pharmaceutically acceptable excipients. In an embodiment, the immediate release microparticles of gamma-hydroxybutyrate have a volume mean diameter D(4,3) of from 10 to 1000 microns. In other embodiments, the immediate release microparticles of gamma-hydroxybutyrate have a volume mean diameter D(4,3) of from 95 to 600 microns. In additional embodiments, the immediate release microparticles of gamma-hydroxybutyrate have a volume mean diameter D(4,3) of from 150 to 400 microns. In one embodiment, their volume mean diameter is about 270 microns.

The immediate release particles of gamma-hydroxybutyrate may include a core and a layer deposited on the core that contains the gamma-hydroxybutyrate. The core may be any particle chosen from the group consisting of:
crystals or spheres of lactose, sucrose (such as Compressuc™ PS from Tereos), microcrystalline cellulose (such as Avicel™ from FMC Biopolymer, Cellet™ from Pharmatrans or Celphere™ from Asahi Kasei), sodium chloride, calcium carbonate (such as Omyapure™ 35 from Omya), sodium hydrogen carbonate, dicalcium phosphate (such as Dicafos™ AC 92-12 from Budenheim) or tricalcium phosphate (such as Tricafos™ SC93-15 from Budenheim);
composite spheres or granules, for example sugar spheres comprising sucrose and starch (such as Suglets™ from NP Pharm), spheres of calcium carbonate and starch (such as Destab™ 90 S Ultra 250 from Particle Dynamics) or spheres of calcium carbonate and maltodextrin (such as Hubercal™ CCG4100 from Huber).

The core can also comprise other particles of pharmaceutically acceptable excipients such as particles of hydroxypropyl cellulose (such as Klucel™ from Aqualon Hercules), guar gum particles (such as Grinsted™ Guar from Danisco), xanthan particles (such as Xantural™ 180 from CP Kelco).

According to a particular embodiment of the invention, the cores are sugar spheres or microcrystalline cellulose spheres, such as Cellets™ 90, Cellets™ 100 or Cellets™ 127 marketed by Pharmatrans, or also Celphere™ CP 203, Celphere™ CP305, Celphere™ SCP 100. In one embodiment, the core is a microcrystalline cellulose sphere. For example, the core may be a Cellets™ 127 from Pharmatrans.

In various embodiments, the core has a mean volume diameter of about 95 to about 450 microns, about 95 to about 170 microns, or about 140 microns.

The layer deposited onto the core comprises the immediate release gamma-hydroxybutyrate. In an embodiment, the layer also comprises a binder, which may be chosen from the group consisting of:
low molecular weight hydroxypropyl cellulose (such as Klucel™ EF from Aqualon-Hercules), low molecular weight hydroxypropyl methylcellulose (or hypromellose) (such as Methocel™ E3 or E5 from Dow), or low molecular weight methylcellulose (such as Methocel™ A15 from Dow);
low molecular weight polyvinyl pyrrolidone (or povidone) (such as Plasdone™ K29/32 from ISP or Kollidon™ 30 from BASF), vinyl pyrrolidone and vinyl acetate copolymer (or copovidone) (such as Plasdone™ S630 from ISP or Kollidon™ VA 64 from BASF);
dextrose, pregelatinized starch, maltodextrin; and mixtures thereof.

Low molecular weight hydroxypropyl cellulose corresponds to grades of hydroxypropyl cellulose having a molecular weight of less than 800,000 g/mol, less than or equal to 400,000 g/mol, or less than or equal to 100,000 g/mol. Low molecular weight hydroxypropyl methylcellulose (or hypromellose) corresponds to grades of hydroxypropyl methylcellulose the solution viscosity of which, for a 2% solution in water and at 20° C., is less than or equal to 1,000 mPa·s, less than or equal to 100 mPa·s, or less than or equal to 15 mPa·s. Low molecular weight polyvinyl pyrrolidone (or povidone) corresponds to grades of polyvinyl pyrrolidone having a molecular weight of less than or equal to 1,000,000 g/mol, less than or equal to 800,000 g/mol, or less than or equal to 100,000 g/mol.

In some embodiments, the binding agent is chosen from low molecular weight polyvinylpyrrolidone or povidone (for example, Plasdone™ K29/32 from ISP), low molecular weight hydroxypropyl cellulose (for example, Klucel™ EF from Aqualon-Hercules), low molecular weight hydroxypropyl methylcellulose or hypromellose (for example, Methocel™ E3 or E5 from Dow) and mixtures thereof.

In one embodiment, the binder is povidone K30 or K29/32, especially Plasdone™ K29/32 from ISP. The binder may be present in an amount of 0 to 80%, 0 to 70%, 0 to 60%, 0 to 50%, 0 to 40%, 0 to 30%, 0 to 25%, 0 to 20%, 0 to 15%, 0 to 10%, or from 1 to 9% of binder based on the total weight of the immediate release coating. In an embodiment, the binder is present in an amount of 5% based on the total weight of the immediate release coating. In one embodiment, the amount of binder is 5% of binder over the total mass of gamma-hydroxybutyrate and binder.

The layer deposited on the core can represent at least 10% by weight, and even greater than 20, 25, 30, 35, 40, 45, 50, 55, 60, 65, 70, 75, 80, 85 or 90% by weight of the total weight of the immediate release particle of gamma-hydroxybutyrate. In one embodiment, the layer deposited on the core represents about 85% of the weight of the immediate release particle of gamma-hydroxybutyrate.

According to an embodiment, the immediate-release particles comprise 80.75% w/w of gamma-hydroxybutyrate, 4.25% w/w of Povidone K30 and 15% of microcrystalline cellulose spheres.

According to another embodiment, the immediate-release particles comprise 80.75% w/w of gamma-hydroxybutyrate, 4.25% w/w of Povidone K30 and 15% of microcrystalline cellulose spheres with a volume mean diameter of about 95 microns to about 450 microns.

According to yet another embodiment, the immediate-release particles comprise 80.75% w/w of gamma-hydroxybutyrate, 4.25% w/w of Povidone K30 and 15% of microcrystalline cellulose spheres with a volume mean diameter of about 95 microns to about 170 microns.

According to an embodiment, the immediate-release particles comprise 80.75% w/w of sodium oxybate, 4.25% w/w of Povidone K30 and 15% of microcrystalline cellulose spheres.

According to another embodiment, the immediate-release particles comprise 80.75% w/w of potassium salt of gamma-hydroxybutyric acid, 4.25% w/w of Povidone K30 and 15% of microcrystalline cellulose spheres.

According to another embodiment, the immediate-release particles comprise 80.75% w/w of calcium salt of gamma-hydroxybutyric acid, 4.25% w/w of Povidone K30 and 15% of microcrystalline cellulose spheres.

According to another embodiment, the immediate-release particles comprise 80.75% w/w of magnesium salt of gamma-hydroxybutyric acid, 4.25% w/w of Povidone K30 and 15% of microcrystalline cellulose spheres.

According to another embodiment, the immediate-release particles are manufactured by dissolving the gamma-hydroxybutyrate and the Povidone K30 in a mixture of water/ethanol 40/60 w/w and spraying the resulting solution onto the surface of the microcrystalline cellulose spheres.

### Other Characteristics of Modified Release Portion

The modified release portion may be any formulation that provides the desired in vitro dissolution profile of gamma-hydroxybutyrate. The modified release portion may include modified release particles, obtained by coating immediate release particles of gamma-hydroxybutyrate with a coating (or coating film) that inhibits the immediate release of the gamma-hydroxybutyrate. In one sub-embodiment the modified release portion comprises particles comprising: (a) an inert core; (b) a coating; and (c) a layer comprising the gamma hydroxybutyrate interposed between the core and the coating.

In an embodiment, the modified release portion comprises a time-dependent release mechanism and a pH-dependent release mechanism.

In an embodiment, the coating film comprises at least one polymer carrying free carboxylic groups, and at least one hydrophobic compound characterized by a melting point equal or greater than 40° C.

The polymer carrying free carboxylic groups may be selected from: (meth)acrylic acid/alkyl (meth)acrylate copolymers or methacrylic acid and methylmethacrylate copolymers or methacrylic acid and ethyl acrylate copolymers or methacrylic acid copolymers type A, B or C, cellulose derivatives carrying free carboxylic groups, cellulose acetate phthalate, cellulose acetate succinate, hydroxypropyl methyl cellulose phthalate, carboxymethylethyl cellulose, cellulose acetate trimellitate, hydroxypropyl methyl cellulose acetate succinate, polyvinyl acetate phthalate, zein, shellac, alginate and mixtures thereof.

In an embodiment, the methacrylic acid copolymers are chosen from the group consisting of poly (methacrylic acid, methyl methacrylate) 1:1 or Eudragit™ L100 or equivalent, poly (methacrylic acid, ethyl acrylate) 1:1 or Eudragit™ L100-55 or equivalent and poly (methacrylic acid, methyl methacrylate) 1:2 or Eudragit™ S100 or equivalent.

In another embodiment the coating comprises a polymer carrying free carboxylic groups wherein the free carboxylic groups are substantially ionized at pH 7.5.

The hydrophobic compound with a melting point equal or greater than 40° C. may be selected from the group consisting of hydrogenated vegetable oils, vegetable waxes, wax yellow, wax white, wax microcrystalline, lanolin, anhydrous milk fat, hard fat suppository base, lauroyl macrogol glycerides, polyglyceryl diisostearate, diesters or triesters of glycerol with a fatty acid, and mixtures thereof.

In various embodiments, the hydrophobic compound with a melting point equal or greater than 40° C. is chosen from the group of following products: hydrogenated cottonseed oil, hydrogenated soybean oil, hydrogenated palm oil, glyceryl behenate, hydrogenated castor oil, candellila wax, tristearin, tripalmitin, trimyristin, yellow wax, hard fat or fat that is useful as suppository bases, anhydrous dairy fats, lanolin, glyceryl palmitostearate, glyceryl stearate, lauryl macrogol glycerides, polyglyceryl diisostearate, diethylene glycol monostearate, ethylene glycol monostearate, omega 3 fatty acids, and mixtures thereof. For example, the hydrophobic compound may include hydrogenated cottonseed oil, hydrogenated soybean oil, hydrogenated palm oil, glyceryl behenate, hydrogenated castor oil, candelilla wax, tristearin, tripalmitin, trimyristin, beeswax, hydrogenated poly-1 decene, carnauba wax, and mixtures thereof.

In practice, and without this being limiting, the hydrophobic compound with a melting point equal or greater than 40° C. may be chosen from the group of products sold under the following trademarks: Dynasan™, Cutina™, Hydrobase™, Dub™, Castorwax™, Croduret™, Compritol™, Sterotex™, Lubritab™, Apifil™, Akofine™, Softisan™, Hydrocote™, Livopol™, Super Hartolan™, MGLA™, Corona™, Protalan™, Akosoft™, Akosol™, Cremao™, Massupol™, Novata™, Suppocire™, Wecobee™, Witepsol™, Lanolin™, Incromega™, Estaram™, Suppoweiss™, Gelucire™, Precirol™, Emulcire™, Plurol diisostéarique™, Geleol™, Hydrine™, Monthyle™, Kahlwax™ and mixtures thereof. In an embodiment, the hydrophobic compound with a melting point equal or greater than 40° C. may be chosen from the group of products sold under the following trademarks: Dynasan™ P60, Dynasan™114, Dynasan™116, Dynasan™118, Cutina™ HR, Hydrobase™ 66-68, Dub™ HPH, Compritol™ 888, Sterotex™ NF, Sterotex™ K, Lubritab™, and mixtures thereof.

A particularly suitable coating is composed of a mixture of hydrogenated vegetable oil and a methacrylic acid copolymer. The exact structure and amount of each component, and the amount of coating applied to the particle, controls the release rate and release triggers. Eudragit® methacrylic acid copolymers, namely the methacrylic acid—methyl methacrylate copolymers and the methacrylic acid—ethyl acrylate copolymers, have a pH-dependent solubility: typically, the pH triggering the release of the active ingredient from the microparticles is set by the choice and mixture of appropriate Eudragit® polymers. In the case of gamma hydroxybutyrate modified release microparticles, the theoretical pH triggering the release is from 5.5 to 6.97 or from 5.5 to 6.9. By “pH trigger” is meant the minimum pH above which dissolution of the polymer occurs.

In a particular embodiment, the coating comprises a hydrophobic compound with a melting point equal or greater than 40° C. and a polymer carrying free carboxylic groups are present in a weight ratio from 0.4 to 4, from 0.5 to 4, from 0.6 to 2.5, from 0.67 to 2.5, from 0.6 to 2.33, or from 0.67 to 2.33. In one embodiment, the weight ratio is about 1.5.

A particularly suitable coating is composed of a mixture of hydrogenated vegetable oil and a methacrylic acid copolymer with a theoretical pH triggering the release from 6.5 up to 6.97 in a weight ratio from 0.4 to 4, from 0.5 to 4, from 0.6 to 2.5, from 0.67 to 2.5, from 0.6 to 2.33, or from 0.67 to 2.33. In one embodiment, the weight ratio may be about 1.5.

The modified release particles of gamma-hydroxybutyrate have a volume mean diameter of from 100 to 1200 microns, from 100 to 500 microns, or from 200 to 800 microns. In one embodiment, the modified release particles of gamma-hydroxybutyrate have a volume mean diameter of about 320 microns.

The coating can represent 10 to 50%, 15 to 45%, 20 to 40%, or 25 to 35% by weight of the total weight of the coated modified release particles. In one embodiment, the coating represents 25-30% by weight of the total weight of the modified release particles of gamma-hydroxybutyrate.

In an embodiment, the coating layer of the modified release particles of gamma-hydroxybutyrate is obtained by spraying, in particular in a fluidized bed apparatus, a solution, suspension or dispersion comprising the coating composition as defined previously onto the immediate release particles of gamma-hydroxybutyrate, in particular the immediate release particles of gamma-hydroxybutyrate as previously described. In one embodiment, the coating is formed by spraying in a fluidized bed equipped with a Wurster or partition tube and according to an upward spray orientation or bottom spray a solution of the coating excipients in hot isopropyl alcohol.

According to an embodiment, the modified release particles of gamma-hydroxybutyrate consist of 10.5% w/w of microcrystalline cellulose spheres with a volume mean diameter of about 95 microns to about 450 microns, layered with 56.5% w/w of gamma-hydroxybutyrate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of gamma-hydroxybutyrate.

According to an embodiment, the modified release particles of gamma-hydroxybutyrate consist of 10.5% w/w of microcrystalline cellulose spheres with a volume mean diameter of about 95 microns to about 170 microns, layered with 56.5% w/w of gamma-hydroxybutyrate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of gamma-hydroxybutyrate.

According to an embodiment, the modified release particles of gamma-hydroxybutyrate consist of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of sodium oxybate.

According to an embodiment, the modified release particles of gamma-hydroxybutyrate consist of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of sodium oxybate.

According to another embodiment, the modified release particles of gamma-hydroxybutyrate consist of 11.3% w/w of microcrystalline cellulose spheres with a volume mean diameter of about 95 microns to about 450 microns, layered with 60.5% w/w of gamma-hydroxybutyrate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

According to another embodiment, the modified release particles of gamma-hydroxybutyrate consist of 11.3% w/w of microcrystalline cellulose spheres with a volume mean diameter of about 95 microns to about 170 microns, layered with 60.5% w/w of gamma-hydroxybutyrate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

According to another embodiment, the modified release particles of gamma-hydroxybutyrate consist of 11.3% w/w of microcrystalline cellulose spheres with a volume mean diameter of about 95 microns to about 450 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

According to another embodiment, the modified release particles of gamma-hydroxybutyrate consist of 11.3% w/w of microcrystalline cellulose spheres with a volume mean diameter of about 95 microns to about 170 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

### Packaging

The composition of gamma-hydroxybutyrate may be supplied in sachets or stick-packs comprising a particulate formulation. The sachets may be available in several different doses, comprising gamma-hydroxybutyrate in amounts equivalents to 0.5 g, 1.0 g, 1.5 g, 3.0 g, 4.5 g, 6.0 g, 7.5 g, 9.0 g, 10.5 g and/or 12 g of sodium oxybate. Depending on the dose required, one or more of these sachets may be opened, and its contents mixed with tap water to provide the nightly dose of gamma-hydroxybutyrate.

### Methods of Treatment

Provided herein are methods for treating a human patient suffering from one or more symptoms of narcolepsy by orally administering a single daily dose to the human patient a full dosage amount of a pharmaceutical composition comprising gamma-hydroxybutyrate concomitantly with divalproex sodium. In some embodiments, the method may be effective to treat a disorder including but not limited to narcolepsy in a human patient in need thereof. Treatment of narcolepsy may include improvement (e.g., reduction) in one or more symptoms such as cataplexy, excessive daytime sleepiness, disrupted nighttime sleep, hypnagogic hallucinations, or sleep paralysis. In some examples, the human patient may be a human subject. Further provided herein is a method of treating a disorder treatable with gamma-hydroxybutyrate in a human subject in need thereof comprising orally administering a single daily dose to the human amounts of gamma-hydroxybutyrate equivalent to from 3.0 to 12.0 g of sodium oxybate in the composition concomitantly with divalproex sodium. Further provided herein are methods of treating narcolepsy, types 1 and/or 2, by orally administering a therapeutically effective amount of a gamma-hydroxybutyrate formulation characterized by the novel gamma-hydroxybutyrate pharmacokinetic properties of the composition when co-administered with divalproex sodium without reducing the dosage of gamma-hydroxybutyrate that would be administered absent the divalproex sodium. In an embodiment, the composition of the present invention is effective to treat narcolepsy Type 1 or Type 2, wherein the treatment of narcolepsy is defined as reducing excessive daytime sleepiness, reducing the frequency of cataplectic attacks, reducing disrupted nighttime sleep, reducing hypnagogic hallucinations, or reducing sleep paralysis. The therapeutically effective amount may include equivalents from 3.0 to 12.0 g of sodium oxybate. In various embodiments, the therapeutically effective amount is 4.5, 6.0, 7.5 or 9.0 g of sodium oxybate. In one embodiment, the therapeutically effective amount is 6 g or 9 g of sodium oxybate. In various embodiments, the formulation includes sodium oxybate present in a unit dose of at least 4.5 g, at least 6.0 g, at least 7.5 g, or at least 9.0 g. The effectiveness of the treatment may be measured by one or any combination of the following criteria:
Increase the mean sleep latency, as determined on the Maintenance of Wakefulness Test (MWT)
Improve the Clinical Global Impression (CGI) rating of sleepiness
Decrease the number of cataplexy attacks (NCA) determined from the cataplexy frequency item in the Sleep and Symptoms Daily Diary
Decrease the disturbed nocturnal sleep (DNS), the disturbed nocturnal events or the adverse respiratory events as determined by polysomnographic (PSG) measures of sleep fragmentation
Decrease the excessive daytime sleepiness (EDS) as measured by patient report via the Epworth Sleepiness Scale (ESS)
Decrease the daytime sleepiness as measured by the Maintenance of Wakefulness Test based on EEG measures of wakefulness
Decrease PSG transitions from N/2 to N/3 and REM sleep to wake and N1 sleep (as determined by C Iber, S Ancoli-Israel, A Chesson, S F Quan. The AA SM Manual for the Scoring of Sleep and Associated Events. Westchester, Ill.: American Academy of Sleep Medicine; 2007).
Decrease the number of arousals or wakenings, obtained from a PSG as defined by the American Academy of Sleep Medicine
Improve the sleep quality, obtained from one or more of (i) the Sleep and Symptom Daily Diary, (ii) Visual Analog Scale (VAS) for sleep quality and sleep diary, and (iii) VAS for the refreshing nature of sleep
Decrease the Hypnagogic Hallucinations (HH) or sleep paralysis (SP) symptoms in NT1 narcolepsy patients, as measured by the Sleep and Symptom Daily Diary

In an embodiment, the treatment using the composition co-administered with divalproex sodium is superior, as measured by any one or combination of the foregoing criteria, to an equal dose of the composition administered without divalproex sodium.

In some examples, the method includes treatment of narcolepsy Type 1 or Type 2 wherein, compared to a dosing regimen consisting of reducing the dosage sodium oxybate when concomitantly administering with divalproex sodium, a single daily dose administration of a therapeutically effective amount of the formulation of the invention concomitantly administered with divalproex sodium has been shown to not require a reduction in the sodium oxybate dosage.

### EXAMPLES

### Example 1. Formulations

Tables 1a-1d provide the qualitative and quantitative compositions of sodium oxybate IR microparticles, MR microparticles, and mixtures of IR and MR microparticles. The physical structure of the microparticles showing the qualitative and quantitative composition of the IR and MR microparticles is depicted in FIG. 1.

Briefly, sodium oxybate immediate release (IR) microparticles were prepared as follows: 1615.0 g of sodium oxybate and 85.0 g of polyvinylpyrrolidone (Povidone K30-Plasdone™ K29/32 from ISP) were solubilized in 1894.3 g of absolute ethyl alcohol and 1262.9 g of water. The solution was entirely sprayed onto 300 g of microcrystalline cellulose spheres (Cellets™ 127) in a fluid bed spray coater apparatus. IR Microparticles with volume mean diameter of about 270 microns were obtained.

Sodium oxybate modified release (MR) microparticles were prepared as follows: 22.8 g of methacrylic acid copolymer Type C (Eudragit™ L100-55), 45.8 g of methacrylic acid copolymer Type B (Eudragit™ S100), 102.9 g of hydrogenated cottonseed oil (Lubritab™), were dissolved in 1542.9 g of isopropanol at 78° C. The solution was sprayed entirely onto 400.0 g of the sodium oxybate IR microparticles described above in a fluid bed spray coater apparatus with an inlet temperature of 48° C., spraying rate around 11 g per min and atomization pressure of 1.3 bar. MR microparticles were dried for two hours with inlet temperature set to 56° C. MR microparticles with mean volume diameter of about 320 microns were obtained.

The finished composition, which contains a 50:50 mixture of MR and IR microparticles calculated on their sodium oxybate content, was prepared as follows: 353.36 g of the above IR microparticles, 504.80 g of the above MR microparticles, 14.27 g of malic acid (D/L malic acid), 6.34 g of xanthan gum (Xantural™ 75 from Kelco), 9.51 g of carrageenan gum (Viscarin™ PH209 from FMC Biopolymer), 9.51 g of hydroxyethylcellulose (Natrosol™ 250 M from Ashland) and 4.51 g of magnesium stearate were mixed. Individual samples of 7.11 g (corresponding to a 4.5 g dose of sodium oxybate with half of the dose as immediate-release fraction and half of the dose as modified release fraction) were weighed.

TABLE 1a
Composition of IR Microparticles

| | | Quantity per |
|---|---|---|
| Component | Function | 2.25 g dose (g) |
| Sodium oxybate | Drug substance | 2.25 |
| Microcrystalline cellulose | Core | 0.418 |
| spheres | | |
| Povidone K30 | Binder and excipient | 0.118 |
| | in diffusion coating | |
| Ethyl alcohol | Solvent | Eliminated during |
| | | processing |
| Purified water | Solvent | Eliminated during |
| | | processing |
| Total | | 2.786 |

TABLE 1b
Composition of MR Microparticles

| | | Quantity per |
|---|---|---|
| Component | Function | 4.5 g dose (g) |
| IR Microparticles | Core of MR | 2.786 |
| | microparticles | |
| Hydrogenated Vegetable Oil | Coating excipient | 0.716 |
| Methacrylic acid Copolymer | Coating excipient | 0.159 |
| Type C | | |
| Methacrylic acid Copolymer | Coating excipient | 0.318 |
| Type B | | |
| Isopropyl alcohol | Solvent | Eliminated during |
| | | processing |
| Total | | 3.981 |

TABLE 1c
Qualitative Finished Composition

| | | Quantity per |
|---|---|---|
| Component | Function | 4.5 g dose (g) |
| MR microparticles | Modified release fraction | 3.981 |
| | of sodium oxybate | |
| IR microparticles | Immediate release | 2.786 |
| | fraction of sodium | |
| | oxybate | |
| Malic acid | Acidifying agent | 0.113 |
| Xanthan gum | Suspending agent | 0.050 |
| Hydroxyethylcellulose | Suspending agent | 0.075 |
| Carrageenan gum | Suspending agent | 0.075 |
| Magnesium stearate | Lubricant | 0.036 |
| Total | | 7.116 |

TABLE 1d
Quantitative finished composition

| | | Quantity per |
|---|---|---|
| Component | Function | 4.5 g dose (g) |
| Sodium oxybate | Drug substance | 4.5 |
| Microcrystalline cellulose spheres | Core | 0.836 |
| Povidone K30 | Binder | 0.237 |
| Hydrogenated Vegetable Oil | Coating excipient | 0.716 |
| Methacrylic acid Copolymer Type C | Coating excipient | 0.159 |
| Methacrylic acid Copolymer Type B | Coating excipient | 0.318 |
| Malic acid | Acidifying agent | 0.113 |
| Xanthan gum | Suspending agent | 0.050 |
| Hydroxyethylcellulose | Suspending agent | 0.075 |
| Carrageenan gum | Suspending agent | 0.075 |
| Magnesium stearate | Lubricant | 0.036 |
| Total | | 7.116 |

### Example 1 bis. Alternative Formulation

An alternative formulation to the formulation described in Example 1 is described in Example 1 bis.

Sodium oxybate immediate release (IR) microparticles were prepared by coating the IR microparticles described in Example 1 with a top coat layer. Microparticles were prepared as follows: 170.0 of hydroxypropyl cellulose (Klucel™ EF Pharm from Hercules) were solubilized in 4080.0 g of acetone. The solution was entirely sprayed onto 1530.0 g of the IR microparticles of Example 1 in a fluid bed spray coater apparatus. IR Microparticles with volume mean diameter of about 298 microns were obtained (see Table 1 bis-a).

Sodium oxybate modified release (MR) microparticles were prepared as described in example 1 (see Table 1b).

The finished composition, which contains a 50:50 mixture of MR and IR microparticles based on their sodium oxybate content, was prepared as follows: 412.22 g of the above IR microparticles, 530.00 g of the above MR microparticles, 29.96 g of malic acid (D/L malic acid), 4.96 g of xanthan gum (Xantural™ 75 from Kelco), 4.96 g of colloidal silicon dioxide (Aerosil™ 200 from Degussa) and 9.92 g of magnesium stearate were mixed. Individual samples of 7.45 g (corresponding to a 4.5 g dose of sodium oxybate with half of the dose in an immediate-release fraction and half of the dose in a modified release fraction) were weighed (see Table 1 bis-b and 1 bis-c).

TABLE 1bis-a
Composition of IR Microparticles

| | | Quantity per |
|---|---|---|
| Component | Function | 2.25 g dose (g) |
| Sodium oxybate | Drug substance | 2.25 |
| Microcrystalline cellulose | Core | 0.418 |
| spheres | | |
| Povidone K30 | Binder and excipient in | 0.118 |
| | diffusion coating | |
| Hydroxypropyl cellulose | Top coat | 0.310 |
| Ethyl alcohol | Solvent | Eliminated during |
| | | processing |
| Purified water | Solvent | Eliminated during |
| | | processing |
| Acetone | Solvent | Eliminated during |
| | | processing |
| Total | | 3.096 |

TABLE 1bis-b
Qualitative Finished Composition

| | | Quantity per |
|---|---|---|
| Component | Function | 4.5 g dose (g) |
| MR microparticles | Modified release fraction | 3.981 |
| | of sodium oxybate | |
| IR microparticles | Immediate release fraction | 3.096 |
| | of sodium oxybate | |
| Malic acid | Acidifying agent | 0.225 |
| Xanthan gum | Suspending agent | 0.037 |
| Colloidal silicon dioxide | Gliding agent | 0.037 |
| Magnesium stearate | Lubricant | 0.075 |
| Total | | 7.451 |

TABLE 1bis-c
Quantitative finished composition

| | | Quantity per |
|---|---|---|
| Component | Function | 4.5 g dose (g) |
| Sodium oxybate | Drug substance | 4.5 |
| Microcrystalline cellulose spheres | Core | 0.836 |
| Povidone K30 | Binder | 0.237 |
| Hydroxypropyl cellulose | Top coat | 0.310 |
| Hydrogenated Vegetable Oil | Coating excipient | 0.716 |
| Methacrylic acid Copolymer Type C | Coating excipient | 0.159 |
| Methacrylic acid Copolymer Type B | Coating excipient | 0.318 |
| Malic acid | Acidifying agent | 0.225 |
| Xanthan gum | Suspending agent | 0.037 |
| Colloidal silicon dioxide | Gliding agent | 0.037 |
| Magnesium stearate | Lubricant | 0.075 |
| Total | | 7.451 |

Compared to the finished composition described in Example 1, this alternative composition has the following characteristics: same MR microparticles, same IR microparticles but with a top coat, increased amount of malic acid, only one suspending agent (xanthan gum) and presence of a glidant.

### Example 2. In Vivo Pharmacokinetic Study of FT218 with and without DVP

Pharmacokinetic testing was undertaken in vivo in healthy human volunteers for a test product with the finished composition of Example 1 (FT218) co-administered with DVP. The study was designed to describe the magnitude of PK changes in FT218 when co-administrated with divalproex sodium ER evening dose. A total of 24 healthy male subjects between 18 and 55 years of age and with a BMI between 19.1 and 28.0 kg/m² participated in the study. One subject withdrew consent on Day 9 (pre-co-administration) and therefore, n=23 were administered FT218 with a 1250 mg/day divalproex sodium ER and n=24 were administered FT218 without DVP.

The study included a sequential, three period design with a single-dose administration of 6 g FT218 on Day 1 (Period 1), once daily 1250 mg divalproex sodium ER administration from Day 2-11 (Period 2), and FT218 and divalproex sodium ER co-administration on Day 12 (Period 3). All administrations were performed in the evening, 2 hours after the completion of dinner. Overall, no major safety issues were observed during this study and no SAEs or AESIs occurred.

Following administration of 6 g FT218 in the evening of Day 1, quantifiable concentrations of GHB were observed after 10 minutes (the first sampling point) for all subjects. Concentrations of GHB increased with maximum geometric mean concentration of 71.2 μg/mL reached at approximately 1 hour after administration. After reaching the peak concentration, GHB concentrations gradually decreased. Plasma concentrations of GHB were quantifiable in all subjects until at least 8 hours postdose.

The concentration versus time curves of FT218 with and without DVP are presented in FIGS. 1A and 1B. The derived PK parameters are summarized below (Table 2).

Co-administration of a single dose of 6 g FT218 with divalproex sodium ER in the evening increased AUC₀₋ₜ and AUC_{0-inf} for GHB by approximately 17%. The 90% CIs of the ratio of the mean of Cₘₐₓ and AUC were contained within the standard bioequivalence range (80.00%-125.00%), respecting the bioequivalence criteria. Cₘₐₓ was not affected by the co-administration of divalproex sodium ER. Tₘₐₓ were comparable with or without co-administration of divalproex sodium ER.

TABLE 2
Mean PK Parameters

| | Tmax (h) | Cmax | AUC₀₋ₗₐₛₜ | AUC_{0-inf} | AUC_{0-8 h} | C8 h | |
|---|---|---|---|---|---|---|---|
| | [min- | (μg/mL) ± | (μg/mL · h) ± | (μg/mL · h) ± | (μg/mL · h) ± | (μg/mL) ± | |
| Treatment | max] | SD (CV) | SD (CV) | SD (CV) | SD (CV) | SD (CV) | |
| FT218 alone | 1.3 [0.3-3.0] | 80 ± 20 (25%) | 307 ± 107 (35%) | 308 ± 107 (35%) | 304 ± 105 (34%) | 4.0 ± 4.3 | (108%) |
| n = 24 | | | | | | | |
| FT218 + DVP | 2.0 [0.3-3.5] | 78 ± 19 (25%) | 366 ± 146 (40%) | 366 ± 146 (40%) | 355 ± 133 (38%) | 9.8 ± 10.7 | (108) |
| n = 23 | | | | | | | |

### Example 3. Comparison of FT218 with and without DVP

To compare the effect of DVP on FT218, the mean values for Tₘₐₓ, Cₘₐₓ, and AUC_{inf} with FT218 alone and FT218 with DVP were plotted together. The effect of DVP on FT218 is shown in FIGS. 2A, 2B, and 2C. FIG. 2A shows the mean Tₘₐₓ values for each patient when administered FT218 alone and when co-administered with DVP. FIG. 2B shows the mean Cₘₐₓ values for each patient when administered FT218 alone and when co-administered with DVP. FIG. 2C shows the mean AUC_{inf} values for each patient when administered FT218 alone and when co-administered with DVP. In comparison, FT218 with DVP appears to demonstrate similar behavior as FT218 alone. Thus, FT218 with and without DVP appear to have similar PK profiles.

The Point Estimate (PE) providing the geometric mean ratio of FT218+DVP/FT218 (alone) and 90% confidence intervals (CI) of the PE are shown below (Table 3). The 90% CIs of the ratio of the mean of Cₘₐₓ and AUC were contained within the standard bioequivalence range (80.00%-125.00%), respecting the bioequivalence criteria. The 90% CI for the T/R ratio did not include 100 for AUC_{0-inf} (T/R ratio [90% CI]: 116.74 [111.03-122.73]) and AUC₀₋ₜ (T/R ratio [90% CI]: 116.67 [111.18-122.44]), indicating an increase of AUC by approximately 17%. Cₘₐₓ for both treatments was similar (T/R ratio [90% CI]: 98.46 [91.58-105.85]). Thus, Cₘₐₓ, AUC₀₋ₗₐₛₜ and AUC_{0-inf} 90% confidence intervals are within the 80-125% bioequivalence range. The tₘₐₓ for GHB was comparable for both treatments. This was confirmed by non-parametric statistical analysis.

TABLE 3
PK Analysis

| PK | PE (ratio geomean) | 90% CI | 90% CI |
|---|---|---|---|
| Parameter | (FT218 + DVP/FT218 alone) | Lower | Upper |
| Cₘₐₓ | 98.46 | 91.58 | 105.85 |
| AUC₀₋ₗₐₛₜ | 116.67 | 111.18 | 122.44 |
| AUC_{0-inf} | 116.52 | 111.07 | 122.23 |

### Example 4. Comparison of DVP with and without FT218

Following administration of 1250 mg divalproex sodium ER in the evening of Day 11, the geometric mean concentration of valproic acid increased from 58.5 μg/mL at baseline to a maximum geometric mean concentration of 79.5 μg/mL 14 hours after administration. After reaching the peak concentration, geometric mean concentrations of valproic acid returned to 57.9 μg/mL at 24 hours after administration.

Following administration of 1250 mg divalproex sodium ER in the evening of Day 12, in the presence of concentrations of GHB, the geometric mean concentration of valproic acid increased from 57.9 μg/mL at baseline to a maximum geometric mean concentration of 77.0 μg/mL 14 hours after administration. After reaching the peak concentration, geometric mean concentrations of valproic acid returned to 64.0 μg/mL at 24 hours after administration.

For subjects who received both divalproex sodium ER treatments on Day 11 (without FT218) and Day 12 (with FT218), respectively, and who were included in the statistical analysis (N=23), the geometric means of Cₘₐₓ and AUC₀₋₂₄ for valproic acid on both days were compared. The 90% CIs of the ratio of the mean of Cₘₐₓ and AUC₀₋₂₄ were contained within the standard bioequivalence range (80.00%-125.00%), respecting the bioequivalence criteria. AUC₀₋₂₄ for both treatments was similar (T/R ratio [90% CI]: 97.28 [94.59-100.04]). For Cₘₐₓ, the 90% CI for the T/R ratio did not include 100 (T/R ratio [90% CI]: 94.82 [91.03-98.76]), indicating a decrease of Cₘₐₓ by approximately 5%. The tₘₐₓ for valproic acid was comparable for both treatments. This was confirmed by non-parametric statistical analysis.

To compare the effect of FT218 on DVP, the concentration versus time curves for a 1250 mg dose of DVP administered alone (Day 11) and co-administered with FT218 (Day 12) were plotted together.

FIG. 3A shows the mean PK profiles of DVP with and without co-administration with FT218. FIG. 3B shows individual PK profiles of DVP with and without co-administration with FT218. This appears to demonstrate a similar DVP profile with or without FT218.

### Example 5. Comparisons with DDI Study of Xyrem®

To compare the effect of DVP on FT218 and Xyrem®, the geometric LS mean AUC_{inf} values for FT218 with and without DVP were compared with the geometric LS mean AUC_{inf} values for Xyrem® with and without DVP from a drug-drug interaction (DDI) study for Xyrem (Eller et al, 2013). Tables 4 and 5 below provide the comparison. Table 4 shows that the Cₘₐₓ and AUC_{inf} for 6 g FT218 with 1250 mg/day DVP are within the 80%-125% bioequivalence range of the Cₘₐₓ and AUC_{inf} for a 6 g dose of FT218 alone, while Table 5 shows the AUC_{inf} for two 3 g doses Xyrem® with 1250 mg/day DVP is above the bioequivalence range for AUC_{inf}. Specifically, Xyrem administered with DVP without adjusting dosage resulted in about 127% AUC_{inf} of Xyrem alone while FT218 administered with DVP without adjusting dosage resulted in about 117% AUC_{inf} of FT218 alone. Thus, Xyrem with DVP is outside bioequivalence limits, while FT218 with DVP is within the bioequivalence limits.

TABLE 4
DDI study, PKFT218-1901 (evening dosing)

| | Geometric LS | Geometric LS |
|---|---|---|
| | mean AUC₀₋ᵢₙₜ | mean Cₘₐₓ |
| Treatment | (μg/mL · h) | (μg/mL) |
| FT218 alone (6 g) | 290.48 | 76.81 |
| n = 23 | | |
| FT218 (6 g) + DVP | 338.46 | 75.62 |
| (1250 mg/day) | | |
| n = 23 | | |
| PE (FT218 DVP/ | 116.52 | 98.46 |
| FT218 alone) (%) | | |

TABLE 5
DDI study Xyrem ®, Eller et al. 2013

| | Geometric LS | Geometric LS |
|---|---|---|
| | mean AUC_{0-inf} | mean Cₘₐₓ |
| Treatment | (μg/mL · h) | (μg/mL) |
| Xyrem ® alone | 275.6 | Not detailed |
| (twice 3 g) | | |
| n = 20 | | |
| Xyrem ® | 349.7 | Not detailed |
| (twice 3 g) + DVP | | |
| (1250 mg/day) | | |
| n = 20 | | |
| PE (Xyrem ® + DVP/ | 126.9 | Not detailed |
| Xyrem ® alone) (%) | | |

### Example 6. In Vivo Pharmacokinetic Study of FT218 with and without DVP Administered in the Morning

Pharmacokinetic testing was undertaken in vivo in healthy human volunteers for a test product with the finished composition of Example 1 (FT218) co-administered with DVP. The study was an open-label, sequential study to assess the drug-drug interaction of divalproex sodium extended release (ER) at steady-state on the FT218 formulation administered at a single 6 g morning dose in healthy volunteers. A total of 22 healthy subjects participated in the study. A total of 22 subjects completed the study as per protocol and 21 subjects were evaluable for the GHB PK statistical analysis. The FT218 was administered in the morning, 2 h post-morning meal, with or without 1250 mg/day divalproex sodium ER.

The study included a sequential, three period design with a single-dose administration of 6 g FT218 on Day 1 (Period 1), once daily 1250 mg divalproex sodium ER administration from Day 2-11 (Period 2), and FT218 and divalproex sodium ER co-administration on Day 12 (Period 3). All administrations were performed in the morning, 2 hours after the completion of a morning meal. Overall, no major safety issues were observed during this study and no SAEs or AESIs occurred.

Following administration of 6 g FT218 in the morning of Day 1, quantifiable concentrations of GHB were observed after 10 minutes (the first sampling point) for all subjects. After reaching the peak concentration, GHB concentrations gradually decreased. Plasma concentrations of GHB were quantifiable in all subjects until at least 8 hours postdose.

The concentration versus time curves of FT218 with and without DVP are presented in FIGS. 4A and 4B. The derived PK parameters are summarized below (Table 6).

The 90% CIs of the ratio of the mean of Cₘₐₓ and AUC were contained within the standard bioequivalence range (80.00%-125.00%), respecting the bioequivalence criteria. Cₘₐₓ was not affected by the co-administration of divalproex sodium ER. Tₘₐₓ was comparable with or without co-administration of divalproex sodium ER.

TABLE 6
Mean PK Parameters

| | Tmax (h) | | Cmax | AUC₀₋ₗₐₛₜ | AUC_{0-inf} | AUC_{0-8 h} | C8 h | |
|---|---|---|---|---|---|---|---|---|
| | [min- | | (μg/mL) ± | (μg/mL · h) ± | (μg/mL · h) ± | (μg/mL · h) ± | (μg/mL) ± | |
| Treatment | max] | | SD (CV) | SD (CV) | SD (CV) | SD (CV) | SD (CV) | |
| FT218 alone | 0.76 | [0.3-3.03] | 107 ± 25 (24%) | 333 ± 113 (34%) | 334 ± 113 (34%) | 332 ± 112 (34%) | 1.70 ± 1.70 | (100%) |
| n = 22 | | | | | | | | |
| FT218 + DVP | 1.0 | [0.33-4.5] | 108 ± 19 (18%) | 403 ± 140 (35%) | 404 ± 140 (35%) | 399 ± 134 (34%) | 5.62 ± 7.23 | (24%) |
| n = 22 | | | | | | | | |

### Example 7. Comparison of FT218 with and without DVP

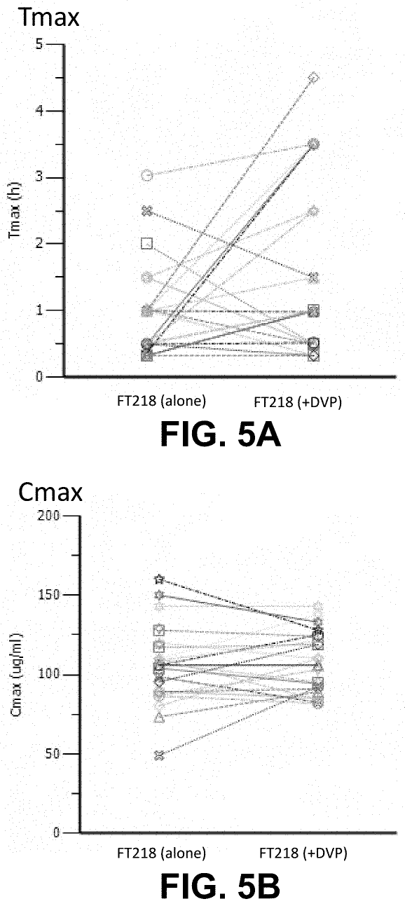

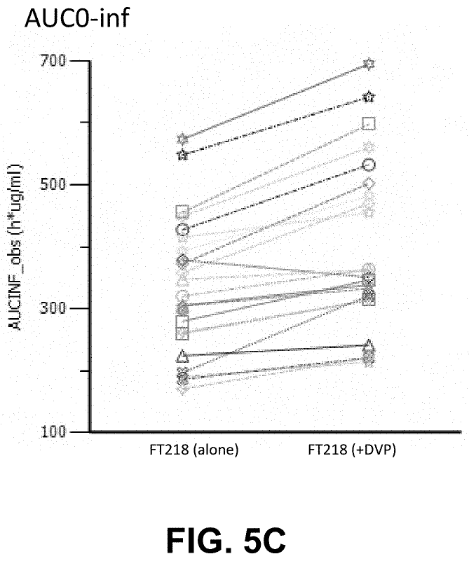

To compare the effect of DVP on FT218 administered once in the morning, the mean values for Tₘₐₓ, Cₘₐₓ, and AUC_{inf} with FT218 alone and FT218 with DVP were plotted together. The effect of DVP on FT218 is shown in FIGS. 5A, 5B, and 5C. FIG. 5A shows the mean Tₘₐₓ values for each patient when administered FT218 alone and when co-administered with DVP. FIG. 5B shows the mean Cₘₐₓ values for each patient when administered FT218 alone and when co-administered with DVP. FIG. 5C shows the mean AUC_{inf} values for each patient when administered FT218 alone and when co-administered with DVP. In comparison, FT218 with DVP appears to demonstrate similar behavior as FT218 alone. Thus, FT218 with and without DVP appear to have similar PK profiles when administered in the morning.

The Point Estimate (PE) providing the geometric mean ratio of FT218+DVP/FT218 (alone) and 90% confidence intervals (CI) of the PE are shown below (Table 7). The 90% CIs of the ratio of the mean of Cₘₐₓ and AUC were contained within the standard bioequivalence range (80.00%-125.00%), respecting the bioequivalence criteria. The results indicate an increase of AUC by approximately 18%. Cₘₐₓ for both treatments was similar. Thus, Cₘₐₓ, AUC₀₋ₗₐₛₜ and AUC_{0-inf} 90% confidence intervals are within the 80-125% bioequivalence range.

TABLE 7
PK Analysis

| PK | PE (ratio geomean) | 90% CI | 90% CI |
|---|---|---|---|
| Parameter | (FT218 + DVP/FT218 alone) | Lower | Upper |
| Cₘₐₓ | 103.93 | 96.11 | 112.49 |
| AUC₀₋ₗₐₛₜ | 118.82 | 113.94 | 123.91 |
| AUC_{0-inf} | 118.76 | 113.88 | 123.84 |

### Example 8. Comparison of DVP with and without FT218

Following administration of 1250 mg divalproex sodium ER in the morning of Day 11, the geometric mean concentration of valproic acid increased to a maximum geometric mean concentration of 72.43 μg/mL.

For subjects who received both divalproex sodium ER treatments on Day 11 (without FT218) and Day 12 (with FT218), respectively, and who were included in the statistical analysis (N=22), the geometric means of Cₘₐₓ and AUC₀₋₂₄ for valproic acid on both days were compared. The 90% CIs of the ratio of the mean of Cₘₐₓ and AUC₀₋₂₄ were contained within the standard bioequivalence range (80.00%-125.00%), respecting the bioequivalence criteria.

To compare the effect of FT218 on DVP, the concentration versus time curves for a 1250 mg dose of DVP administered alone (Day 11) and co-administered with FT218 (Day 12) were plotted together.

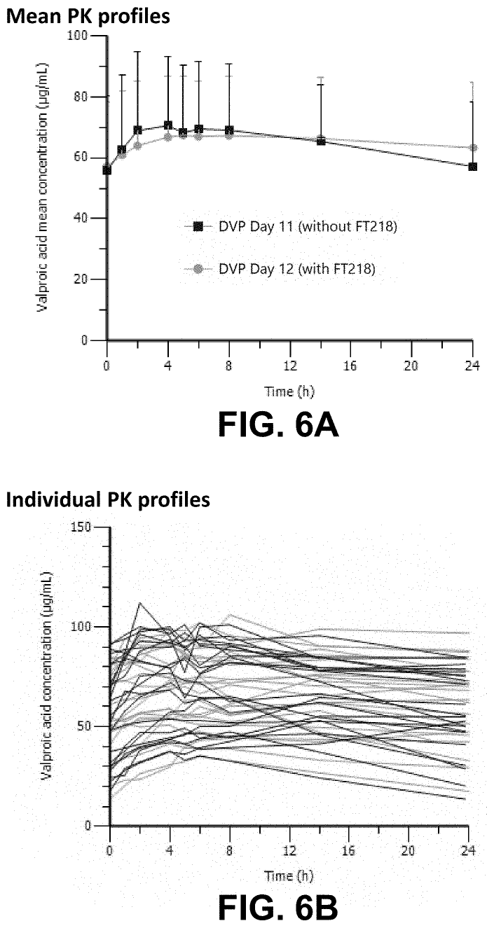

FIG. 6A shows the mean PK profiles of DVP with and without co-administration with FT218 in the morning. FIG. 6B shows individual PK profiles of DVP with and without co-administration with FT218 in the morning. This appears to demonstrate a similar DVP profile with or without FT218.

### Example 9. Inter-Study Comparison of FT218 Alone and with DVP

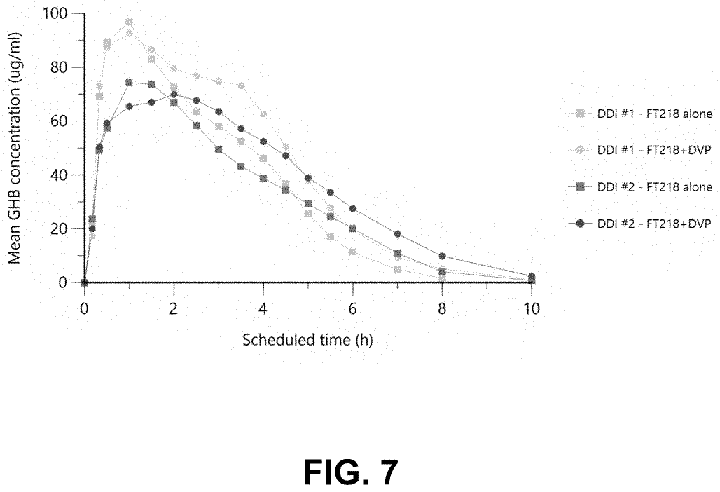

FIG. 7 shows a mean concentration versus time curve for FT218 administered alone and with DVP in two separate studies (DDI #1, DDI #2).

DDI #1 was an open-label, sequential study to assess the drug-drug interaction of divalproex sodium extended release (ER) at steady-state on the FT218 formulation administered at a single 6 g morning dose in healthy volunteers. In DDI #1, the FT218 was administered in the morning, 2 h post-morning meal, with or without 1250 mg/day divalproex sodium ER. Examples 6-8 show the results from DDI #1.

DDI #2 was an open-label, sequential study to assess the drug-drug interaction of divalproex sodium extended release (ER) at steady-state on the FT218 formulation administered at a single 6 g evening dose in healthy male volunteers. In DDI #2, the FT218 was administered in the evening, 2 h post-evening meal, with or without 1250 mg/day divalproex sodium ER. Examples 2-4 show the results from DDI #2.

As seen in FIG. 7, the comparison of DDI #1 and DDI #2 shows that the interaction between FT218 and DVP has a similar effect on the GHB concentration, independent of time of administration. Therefore, FT218 may be co-administered once daily (morning or evening) with DVP without having to reduce the FT218 dosage.

Throughout this application, various publications are referenced. The disclosures of these publications in their entireties are hereby incorporated by reference into this application in order to more fully describe the state of the art to which this invention pertains. It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for treating a patient suffering from one or more symptoms of narcolepsy, the method comprising:
orally administering to the patient a full dosage amount of a pharmaceutical composition comprising gamma-hydroxybutyrate (GHB); and
concomitantly administering a dosage of divalproex sodium (DVP),
wherein the dosage of the GHB composition is not reduced in response to the concomitant administration of DVP.

2. The method of claim 1, wherein the concomitant administration of GHB and DVP provides a substantially bioequivalent PK profile as compared to administration of an equal dose of the GHB composition in the absence of the concomitant administration of DVP.

3. The method of claim 1, wherein the GHB composition is administered once daily.

4. The method of claim 1, wherein a 4.5 g, 6 g, 7.5 g, or 9 g dose of the GHB composition is administered.

5. The method of claim 1, wherein the dosage of DVP is a full dosage of DVP.

6. The method of claim 1, wherein the DVP is administered up to a maximum daily dose of 60 mg/kg/day.

7. The method of claim 1, wherein the dosage of the DVP is not reduced in response to the concomitant administration of GHB composition.

8. The method of claim 1, wherein the concomitant administration of GHB and DVP provides a Cₘₐₓ, AUC₀₋ₗₐₛₜ and/or AUC_{inf} within 80% to 125% of the Cₘₐₓ, AUC₀₋ₗₐₛₜ and/or AUC_{inf} when GHB is administered in the absence of DVP.

9. The method of claim 1, wherein concomitant administration of the GHB composition with divalproex sodium results in a less than 25% mean increase in systemic exposure to the GHB composition.

10. The method of claim 1, wherein concomitant administration of the GHB composition with divalproex sodium results in no change in systemic exposure to the GHB composition.

11. The method of claim 1, wherein the concomitant administration of DVP and a 6 g dosage of the GHB composition provides a mean Cₘₐₓ of 59 μg/mL to 97 μg/mL.

12. The method of claim 1, wherein the concomitant administration of DVP and a 6 g dosage of the GHB composition provides a mean AUC₀₋ₗₐₛₜ of 220 μg/mL·h to 512 μg/mL·h.

13. The method of claim 1, wherein the concomitant administration of DVP and a 6 g dosage of the GHB composition provides a mean AUC_{inf} of 220 μg/mL·h to 512 μg/mL·h.

14. The method of claim 1, wherein the concomitant administration of DVP and a 6 g dosage of the GHB composition provides a mean Tₘₐₓ of 0.3 h to 3.5 h.

15. The method of claim 1, wherein there is no significant reduction in safety or efficacy to a patient following concomitant administration.

16. The method of claim 1, wherein the one or more symptoms of narcolepsy is selected from excessive daytime sleepiness (EDS), disrupted nighttime sleep (DNS), cataplexy, hypnagogic hallucinations, and sleep paralysis.

17. A method for treating a patient suffering from one or more symptoms of narcolepsy, the method comprising:
orally administering to the patient a full dosage amount of a pharmaceutical composition comprising gamma-hydroxybutyrate (GHB); and
concomitantly administering a dosage of divalproex sodium (DVP),
wherein the dosage of GHB is reduced by less than 5% in response to the concomitant administration of DVP.

18. The method of claim 17, wherein the concomitant administration of GHB and DVP provides a substantially bioequivalent PK profile as compared to administration of an equal dose of the GHB composition in the absence of the concomitant administration of DVP.

19. The method of claim 17, wherein the GHB composition is administered once daily.

20. The method of claim 17, wherein a 4.5 g, 6 g, 7.5 g, or 9 g dose of the GHB composition is administered.

21. The method of claim 17, wherein the dosage of DVP is a full dosage of DVP.

22. The method of claim 17, wherein the DVP is administered up to a maximum daily dose of 60 mg/kg/day.

23. The method of claim 17, wherein the dosage of the DVP is not reduced in response to the concomitant administration of GHB composition.

24. The method of claim 17, wherein the concomitant administration of GHB and DVP provides a Cₘₐₓ, AUC₀₋ₗₐₛₜ and/or AUC_{inf} within 80% to 125% of the Cₘₐₓ, AUC₀₋ₗₐₛₜ and/or AUC_{inf} when GHB is administered in the absence of DVP.

25. The method of claim 17, wherein concomitant administration of the GHB composition with divalproex sodium results in a less than 25% mean increase in systemic exposure to the GHB composition.

26. The method of claim 17, wherein concomitant administration of the GHB composition with divalproex sodium results in no change in systemic exposure to the GHB composition.

27. The method of claim 17, wherein the concomitant administration of DVP and a 6 g dosage of the GHB composition provides a mean Cₘₐₓ of 59 μg/mL to 97 μg/mL.

28. The method of claim 17, wherein the concomitant administration of DVP and a 6 g dosage of the GHB composition provides a mean AUC₀₋ₗₐₛₜ of 220 μg/mL·h to 512 μg/mL·h.

29. The method of claim 17, wherein the concomitant administration of DVP and a 6 g dosage of the GHB composition provides a mean AUC_{inf} of 220 μg/mL·h to 512 μg/mL·h.

30. The method of claim 17, wherein the concomitant administration of DVP and a 6 g dosage of the GHB composition provides a mean Tₘₐₓ of 0.3 h to 3.5 h.

31. The method of claim 17, wherein there is no significant reduction in safety or efficacy to a patient following concomitant administration.

32. The method of claim 17, wherein the one or more symptoms of narcolepsy is selected from excessive daytime sleepiness (EDS), disrupted nighttime sleep (DNS), cataplexy, hypnagogic hallucinations, and sleep paralysis.

33. A method for treating a patient suffering from one or more symptoms of narcolepsy, the method comprising:
orally administering to the patient a full dosage amount of a pharmaceutical composition comprising gamma-hydroxybutyrate (GHB); and
concomitantly administering a dosage of divalproex sodium (DVP),
wherein the concomitant administration of GHB and DVP provides a substantially bioequivalent PK profile as compared to administration of an equal dose of the GHB composition in the absence of the concomitant administration of DVP.

34. The method of claim 33, wherein the GHB composition is administered once daily.

35. The method of claim 33, wherein a 4.5 g, 6 g, 7.5 g, or 9 g dose of the GHB composition is administered.

36. The method of claim 33, wherein the dosage of DVP is a full dosage of DVP.

37. The method of claim 33, wherein the dosage of the DVP is not reduced in response to the concomitant administration of GHB composition.

38. The method of claim 33, wherein the DVP is administered up to a maximum daily dose of 60 mg/kg/day.

39. The method of claim 33, wherein the concomitant administration of GHB and DVP provides a Cₘₐₓ, AUC₀₋ₗₐₛₜ and/or AUC_{inf} within 80% to 125% of the Cₘₐₓ, AUC₀₋ₗₐₛₜ and/or AUC_{inf} when GHB is administered in the absence of DVP.

40. The method of claim 33, wherein concomitant administration of the GHB composition with divalproex sodium results in a less than 25% mean increase in systemic exposure to the GHB composition.

41. The method of claim 33, wherein concomitant administration of the GHB composition with divalproex sodium results in no change in systemic exposure to the GHB composition.

42. The method of claim 33, wherein the concomitant administration of DVP and a 6 g dosage of the GHB composition provides a mean Cₘₐₓ of 59 μg/mL to 97 μg/mL.

43. The method of claim 33, wherein the concomitant administration of DVP and a 6 g dosage of the GHB composition provides a mean AUC₀₋ₗₐₛₜ of 220 μg/mL·h to 512 μg/mL·h.

44. The method of claim 33, wherein the concomitant administration of DVP and a 6 g dosage of the GHB composition provides a mean AUC_{inf} of 220 μg/mL·h to 512 μg/mL·h.

45. The method of claim 33, wherein the concomitant administration of DVP and a 6 g dosage of the GHB composition provides a mean Tₘₐₓ of 0.3 h to 3.5 h.

46. The method of claim 33, wherein there is no significant reduction in safety or efficacy to a patient following concomitant administration.

47. The method of claim 33, wherein the one or more symptoms of narcolepsy is selected from excessive daytime sleepiness (EDS), disrupted nighttime sleep (DNS), cataplexy, hypnagogic hallucinations, and sleep paralysis.

48. An oral pharmaceutical composition for the treatment of one or more symptoms of narcolepsy comprising gamma-hydroxybutyrate (GHB) suitable for concomitant administration with a dose of divalproex sodium (DVP).

49. The oral pharmaceutical composition of claim 48, wherein the dosage of GHB is reduced by less than 5% in response to the concomitant administration of DVP.

50. The oral pharmaceutical composition of claim 48, wherein the dosage of GHB is not reduced in response to the concomitant administration of DVP.

51. The oral pharmaceutical composition of claim 48, wherein the dosage of the DVP is not reduced in response to the concomitant administration of GHB composition.

52. The oral pharmaceutical composition of claim 48, wherein concomitant administration of GHB and DVP provides a substantially bioequivalent PK profile as compared to administration of an equal dosage of the GHB composition in the absence of the concomitant administration of DVP.

53. The oral pharmaceutical composition of claim 48, wherein the concomitant administration of GHB and DVP provides a Cₘₐₓ, AUC₀₋ₗₐₛₜ and/or AUC_{inf} within 80% to 125% of the Cₘₐₓ, AUC₀₋ₗₐₛₜ and/or AUC_{inf} when the GHB composition is administered in the absence of DVP.

54. The oral pharmaceutical composition of claim 48, wherein concomitant administration of the GHB composition with divalproex sodium results in a less than 25% mean increase in systemic exposure to the GHB composition.

55. The oral pharmaceutical composition of claim 48, wherein concomitant administration of the GHB composition with divalproex sodium results in no change in systemic exposure to the GHB composition.

56. The oral pharmaceutical composition of claim 48, wherein the GHB composition is suitable for once-daily administration.

57. The oral pharmaceutical composition of claim 48, wherein the GHB composition is administered as a once-daily 4.5 g, 6 g, 7.5 g, or 9 g dose.

58. The oral pharmaceutical composition of claim 48, wherein the DVP is administered up to a maximum daily dose of 60 mg/kg/day.

59. The oral pharmaceutical composition of claim 48, wherein the dosage of DVP is a full dosage of DVP.

60. The oral pharmaceutical composition of claim 48, wherein the concomitant administration of DVP and a 6 g dosage of the GHB composition provides a mean Cₘₐₓ of 59 μg/mL to 97 μg/mL.

61. The oral pharmaceutical composition of claim 48, wherein the concomitant administration of DVP and a 6 g dosage of the GHB composition provides a mean AUC₀₋ₗₐₛₜ of 220 μg/mL·h to 512 μg/mL·h.

62. The oral pharmaceutical composition of claim 48, wherein the concomitant administration of DVP and a 6 g dosage of the GHB composition provides a mean AUC_{inf} of 220 μg/mL·h to 512 μg/mL·h.

63. The oral pharmaceutical composition of claim 48, wherein the concomitant administration of DVP and a 6 g dosage of the GHB composition provides a mean Tₘₐₓ of 0.3 h to 3.5 h.

64. The oral pharmaceutical composition of claim 48, wherein there is no significant reduction in safety or efficacy to a patient following concomitant administration.

65. The oral pharmaceutical composition of claim 48, wherein the composition includes no risk evaluation and mitigation strategy (REMS) program instructions.

66. The oral pharmaceutical composition of claim 48, wherein the composition includes no monitoring instructions for drug drug interactions with gamma-hydroxybutyrate (GHB) and divalproex sodium (DVP).

67. The oral pharmaceutical composition of claim 48, wherein the one or more symptoms of narcolepsy is selected from excessive daytime sleepiness (EDS), disrupted nighttime sleep (DNS), cataplexy, hypnagogic hallucinations, and sleep paralysis.
